(19) United States
(12) Patent Application Publication
(10) Pub. No.: US 2011/0178409 A1
(43) Pub. Date: Jul. 21, 2011

(54) Optical Element

(75) Inventors: Martin Russell Harris, Windsor (AU); Gavan Edmund Rosman, Camberwell (AU); Roderick William Charles Vance, Blackburn (AU); John David Allen, Essendon (AU)

(73) Assignee: OPTISCAN PTY LTD, Victoria (AU)

(21) Appl. No.: 10/585,565

(22) PCT Filed: Feb. 28, 2005
(86) PCT No.: PCT/AU2005/000257

§ 371 Date: May 5, 2009

(30) Foreign Application Priority Data
Feb. 27, 2004 (AU) 2004900986

(51) Int. Cl. A61B 1/06; G02B 21/06; A61B 6/00

(52) U.S. Cl. 600/476; 359/389; 600/163

(57) ABSTRACT

A microscope, endoscope or optical coherence tomograph, comprising a light source, a flexible light transmitter (18) for receiving and transmitting light from the light source, an optical element (20) with a forward end (30) for receiving the light from the light transmitter (18) and a rear wall (26) having an internal surface for reflecting the light laterally, and an external sleeve (14) enclosing the optical element (20) and transparent to the light in at least a region of the sleeve where the light is directed by the internal surface (26). The internal surface (26) has an optical figure suitable for focussing the light to an observational field (28) external to the sleeve (14). A confocal configuration may be used.

### RELATED APPLICATION

This application is based on and claims the benefit of the filing date of AU patent application no. 2004900986 filed 27 Feb. 2004, the contents of which are incorporated herein by reference in its entirety.

### FIELD OF THE INVENTION

The present invention relates to a microscope, endoscope and optical coherence tomograph, and to an optical element for use therein, for viewing laterally, of particular but by no means exclusive application in a confocal probe as, well as in microscopy, endoscopy (including endomicroscopy), colonoscopy, gastroscopy, optical coherence tomography and like applications and especially confocal implementations and/or multi-photon variants of these.

### BACKGROUND OF THE INVENTION

Considerable advances in imaging the interior of the human body have prompted increased interest in the possibility of in vivo microscopic analysis. Confocal microscopy using light returned from fluorescence in tissue can be achieved in an endoscope with endoscope-head diameters around 5 mm with forward looking optics and a vibrating fibre scanner.

Further, probably the most labour intensive component in the existing flexible endoscopes are the as many as 11 element micro-lens assemblies. These lenses are so small that they are difficult to manufacture and handle, including being provided with antireflective coatings after grinding and polishing. It is estimated that perhaps 200 separate operations are required to produce a completed lens assembly.

In addition, the second most expensive component in some prior art systems is the tuning-fork scanning mechanism.

There thus remains a need for still smaller and less expensive devices, in particular so that access can be gained to more remote and at present inaccessible regions of the body, including the internal surfaces of blood vessels.

### SUMMARY OF THE INVENTION

In a first broad aspect, therefore, the present invention provides a microscope or endoscope, comprising;
a light source;
a flexible light transmitter for receiving and transmitting light from said light source;
an optical element with a forward end for receiving said light from said light transmitter and a rear wall having an internal Surface for reflecting said light laterally; and
an external sleeve enclosing said optical element and transparent to said light in at least a region of said external sleeve where said light is directed by said internal surface;
wherein said internal surface has an optical figure suitable for focussing said light to a point observational field external to said external sleeve.

As will be appreciated by the skilled person, the forward end of the optical element may be proximal to or distal from the light source, and that the internal surface may also constitute the rear wall (and, indeed, a portion of a side wall).

In a particular embodiment, the microscope or endoscope is a confocal microscope or endoscope.

In one embodiment, the optical element is rotatable, and said microscope or endoscope includes a drive coupled to said optical element, for rotating said optical element so that said point observational field can be scanned.

In one particular embodiment, the drive comprises an electrical motor located external to said optical element and adjacent to said rear surface of said optical element.

In another particular embodiment, the drive comprises an inner sleeve for supporting and rotating said optical element, whereby said light can be scanned relative to a sample by rotating said inner sleeve and thereby the direction in which said light is focussed by said internal surface. In this embodiment, the drive will typically also include an electrical or other motor for rotating the inner sleeve, though in principle this could be done manually.

In one embodiment, the internal surface is a section of an ellipsoid, whereby the optical element is adapted to receive said light from a substantially point source.

In another embodiment, the internal surface is a section of a paraboloid, whereby the optical element is adapted to receive light in substantial parallel form.

Alternatively, said optical element includes at least one further optical element for changing said light from a divergent beam into a substantial parallel beam, and the internal surface is a section of a paraboloid.

The internal surface can, optionally, have a reflective coating to increase the efficiency of reflection.

In one embodiment, the optical element is oriented with the rear wall proximal to said light source. In this embodiment, the flexible light transmitter may include a bend in order to direct said light towards said internal surface, or the microscope or endoscope may include a mirror for receiving said light from said flexible light transmitter and directing said light towards said internal surface of said optical element.

In a second broad aspect, the present invention provides a optical head for an endoscope or microscope, comprising:
an optical element with a forward end for admitting light from a light source, and a rear wall having an internal surface for reflecting said light laterally, wherein said internal surface has an optical figure suitable for focussing said light; and
an external sleeve enclosing said optical element and transparent to said light in at least a region of said external sleeve where said light is directed by said internal surface.

In one embodiment, the optical element is rotatable, and said head includes a drive coupled to said optical element, for rotating said optical element so that said point observational field can be scanned.

In a third broad aspect, the present invention provides an optical element comprising:
a forward end for admitting light from a light source;
a rear wall having an internal surface for reflecting said light laterally; and
an external sleeve of non-uniform thickness;
wherein said internal surface has an optical figure suitable for focussing said light to a point observational field outside said external sleeve, said external sleeve is rotatable, translatable or both rotatable and translatable relative to said optical element whereby the distance of said point from paid external sleeve can be varied, and said external sleeve is transparent to said light in at least the region where said light is directed by said internal surface.

In each of the above embodiments, the microscope or endoscope may further include a short coherence length light source and a beamsplitter, for performing simultaneous optical coherence tomography.

In each of the above embodiments, the external sleeve may be composed—at least where it must transmit said light and any return light—a polymer with a refractive index close to that of an expected specimen (such as, in some biological applications, PTFE, FEP, ITFE or PFA). Further, the optical element may be, filled with a liquid that has a refractive index that matches, or is close to, the refractive index of the tissue under examination, in order to minimize refraction of light rays in passing from the interior of the block to a point observational field within a specimen.

In another broad aspect, therefore, the present invention provides a optical coherence tomograph, comprising:
a short coherence length light source;
a flexible light transmitter for receiving and transmitting light from said light source;
an optical element with a forward end for receiving said light from said light transmitter and a rear wall having an internal surface for reflecting said light laterally; and
an external sleeve enclosing said optical element and transparent to said light in at least a region of said external sleeve where said light is directed by said internal surface;
wherein said internal surface has an optical figure suitable for focussing said excitation light to an observational field external to said external sleeve.

The invention also provides a method of performing microscopy, endoscopy or optical coherence tomography, comprising:
locating an optical element in or adjacent a specimen, the optical element having a forward end for receiving excitation light from a light transmitter and a rear wall having an internal surface for reflecting said excitation light laterally with an optical figure suitable for focussing said excitation light to a point observational field in or on said specimen;
directing excitation light against said internal surface and thereby to said point observation field; and
collecting by means of said internal surface return light emitted from said specimen in response to said excitation light.

In one embodiment, the optical element is located in an external sleeve that is transparent to said light in at least a region of said sleeve where said light is directed by said internal surface.

In another embodiment, the method includes rotating said optical element, thereby effecting scanning.

All of the above-described aspects and embodiments of the invention may be provided with or as a hand-held probe for examining the exterior or interior of larger specimens (such as samples of meat and the like).

### BRIEF DESCRIPTION OF THE DRAWINGS

In order that the invention may be more clearly ascertained, embodiments will now be described, by way of example, with reference to the accompanying drawings, in which:

FIG. 1 is a view of a scanning head for an endoscope according to an embodiment of the present

FIG. 2A is a schematic view of the optics of the scanning head of FIG. 1;

FIGS. 2B and 2C are schematic plots of the optics of the scanning head of FIG. 1;

FIG. 3 is a view of a scanning head with variable depth for an endoscope according to a further embodiment of the present invention;

FIG. 4 is a partial view of another scanning head with variable depth for an endoscope according to a further embodiment of the present invention;

FIG. 5 is a view of a scanning device for an endoscope according to a further embodiment of the present invention, in which the beam is collimated by means of an achromatic lens and the collimated beam is focussed by means of a paraboloid mirror;

FIG. 6 is a view of a scanning device for an endoscope according to a still further embodiment of the present invention, in which the beam is collimated by means of a pair of on-axis mirrors and the collimated beam is focussed by means of a paraboloid mirror;

FIG. 7 is a view of a scanning device for an endoscope according to a variation of the device of FIG. 6, in which the fibre is off-axis, the beam is collimated by means of a pair of off-axis mirrors and the collimated beam is focussed by means of a paraboloid mirror;

FIGS. 8A and 8B are schematic views of scanning by means of the principal mirror of the embodiment of FIG. 6;

FIGS. 9A and 9B are views of cylindrical elements for use with the embodiments of FIGS. 5 to 7, for correcting astigmatism;

FIG. 10 is a schematic illustration of how variable magnification can be effected by controlling fibre orientation with the embodiments of FIGS. 1, 3 and 4 according to the present invention;

FIG. 11 is a plot of θ₂ (the angle between the projected ray through second focus and the major axis) against θ₁ (the angle between a ray launched from first focus and the major axis) for FIG. 10;

FIG. 12 is a schematic view of a scanning confocal endomicroscope according to another embodiment of the present invention;

FIG. 13A is a schematic view of a scanning confocal endomicroscope and OCT imaging system according to yet another embodiment of the present invention;

FIG. 13B is a schematic view of the optical head region of the scanning confocal endomicroscope and OCT imaging system of FIG. 13A;

FIG. 14A is a schematic view of a scanning confocal endomicroscope according to another embodiment of the present invention;

FIG. 14B is a more detailed view of the optical head region of the system of FIG. 14A;

FIG. 14C is a more detailed view of the region of the motor and belt drive of the system of FIG. 14A;

FIG. 15A is a schematic view of a first portion of a scanning confocal endomicroscope according to another embodiment of the present invention;

FIG. 15B is a schematic view of a second portion of the scanning confocal endomicroscope of FIG. 15A;

FIG. 15C is a more detailed view of the optical head region of the system of FIG. 15A;

FIG. 16 is a schematic view of a scanning confocal endomicroscope according to still another embodiment of the present invention;

FIG. 17A is a schematic view of a scanning confocal endomicroscope and OCT imaging system according to yet another embodiment of the present invention;

FIG. 17B is a schematic view of the optical head region of the scanning confocal endomicroscope and OCT imaging system of FIG. 17A;

FIG. 18 is a schematic view of a scanning confocal microscope according to a further embodiment of the invention;

FIG. 19A is a schematic view of a scanning confocal endomicroscope 430 according to still another embodiment of the present invention;

FIG. 19B is an enlarged view of the optical head of the endomicroscope of FIG. 19A;

FIG. 20A is a schematic view of a scanning optical head for use in various embodiments of the present invention;

FIG. 20B is a schematic view of the optical head of FIG. 20B following activation;

FIG. 21A is a schematic view of a hydraulic apparatus for effecting z motion in various embodiments of the present invention;

FIG. 21B is an enlarged view of the driving coil of the apparatus of FIG. 21A;

FIG. 22 is a schematic view of a hydraulic apparatus for effecting Y motion in various embodiments of the present invention;

FIG. 23A is a schematic view of a scanning optical head for use in various embodiments of the present invention;

FIG. 23B is a schematic view of the optical head of FIG. 20B following activation; and

FIG. 24 is schematic view of an apparatus for providing a fast resonant oscillatory scan in various embodiments of the present invention.

### DETAILED DESCRIPTION

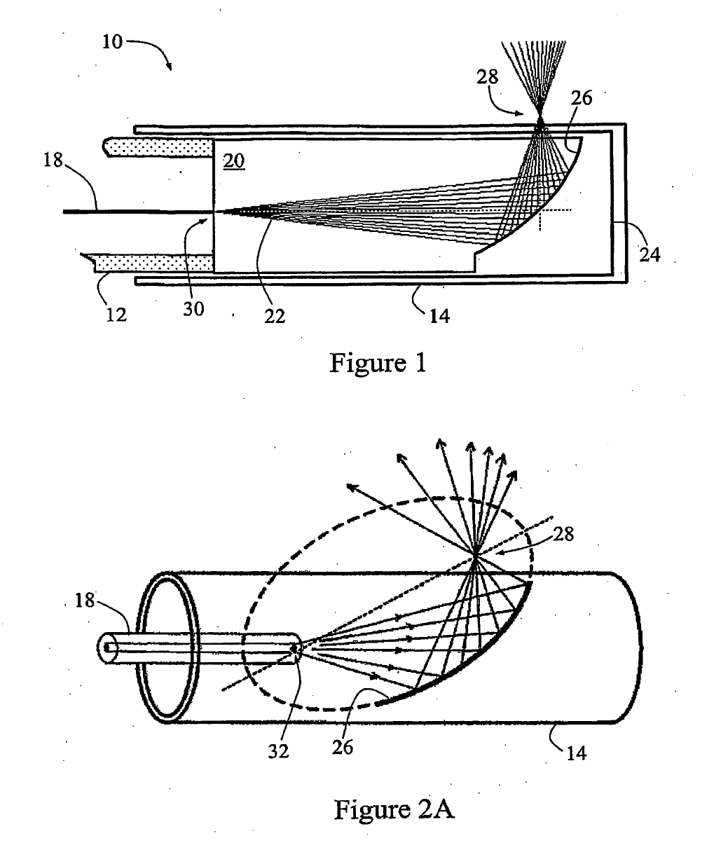

Referring to FIG. 1, according to a first embodiment of the present invention there is provided a scanning, optical head 10 for a confocal endoscope.

The head 10 has two flexible polymer tubes: a rotating inner tube 12 and a fixed cylindrical outer sleeve 14. The inner tube 12 is chosen for torsional stiffness and low coefficient of friction for contact with the external sleeve 14. The optical fibre 18 of the endoscope fits loosely within the inner tube 12, and—including its coating—has a diameter of approximately 250 micron. The inner diameter of the rotating inner tube 12 is about 1 mm; its external diameter is around 1.8 mm. The configuration of inner tube 12 with loose fibre is typical of patch cords used in optical communications systems.

The head 10 also includes an optical element in the form of a generally cylindrical plastic optical block 20, mounted on and co-rotating with inner tube 12. Optical block 20 is composed of either glass or a suitable polymer such as PMMA. In addition, the material of outer sleeve 14 may be composed of a polymer (such as PTFE, PEP, ITFE or PFA) with a refractive index close to that of the specimen which, with many biological specimens, is commonly around 1.33 to 1.38. Suitable polymers have been developed to match tissue refractive indexes, for use in microscopy. In addition, if desired optical block 20 may be filled with a liquid that has a refractive index that matches, or is close to, the refractive index of the tissue under examination and the material of outer sleeve 14, in order to minimize refraction of light rays in passing from the interior of the block 20 to a point observational field within the specimen.

Light 22 (from the laser source of the endoscope) is admitted into the block 20 from the exit of the core of fibre 18, and fans out according to the numerical aperture (NA) of the fibre from the exit point into the block 20. The end wall 24 of block 20 has an internal surface 26 that reflects light 22 laterally. The internal surface 26 is a section of an ellipsoid, aligned so that the divergent beam of light 22 is reflected laterally and focussed by the internal surface 26. Indeed, the light is focussed to a point 28 outside the sleeve 14, which is transparent at least in the region where it must transmit the light. Thus, point 28 represents a point observation field for the endoscope.

Aspheric internal surface 26 is preferably coated to maximize reflection, and can be produced very inexpensively by polymerizing plastic in moulds formed by single point diamond turning on a lathe.

It should be noted that the central ray from the fibre 18 reflects at right angles from the internal surface 26.

In assembly of the head 10, the fibre 18 is attached to the optical block 20 and then the block in attached to the inner tube 12. During this latter operation, any excess fibre is taken up inside the loosely fitting tube 12. It should be noted that the relationship between tube 12 and block 18 need not have optical precision in the sense of focal point accuracy. The main concern is positioning the exit tip of fibre 18 relative to the reflecting internal surface 26, which has a strict relationship to a single point on the block endface. It should also be noted that this fibre attachment point need not be on the optical centreline of the block 20. This design point could be off-centre, anywhere within the inner diameter of the rotating tube 12, provided that the fibre 18 can still be attached in the design position.

Rotation of the inner tube 12 with the optical block 20 attached causes the scanning spot to move around the circumference of the outer sleeve 14 just beyond its outer surface. This rotation can be effected by any suitable means. During this rotation one can gather data concerning the environment through which the focal spot moves, without necessarily producing an image. One application that is envisaged is the examination or at least characterization of plaque cells in blood vessels: as the observational point is rotated, a fluorescence signature may be observed from the blood vessel as the outer sleeve 14 is drawn or pushed along the vessel. Statistics could then be obtained on the types of plaque as a function of length.

On the other hand, gradual longitudinal movement of the inner tube 12 alone during its rotation could gather information for an image if the torsional rigidity of the inner tube is sufficient. The display could then be synchronised with the rotation of the inner tube at the proximal end. One simple method would be the use of a fine thread on the drive mechanism of the inner tube 12.

A simple aide-viewing endoscope is thus provided with an external diameter of as little as 2 to 3 mm. The block 20 is a particularly simple one-piece element that forms an interface between fibre tip and focal point in the tissue which occurs just outside the outer surface of endoscope tip.

Confocal operation is generally associated with an effective pin-hole for transmission and reception and the use of refractive optics as in a normal optical microscope. In the embodiment of FIG. 1, light is emitted from the small core of the optical fibre with the light returning from the focal point back through the fibre for analysis at the proximal end. The fibre end-face acts as both launch and receive pin-hole.

Optical Considerations

The factors leading to the reflective design of this embodiment, as compared to prior art refractive configurations, are as follows. Considering the complexity of multi-element lens designs it may seem that the simple block design of the present embodiment may lack performance. However, as optical rays travel in straight lines within the block 20, the need for complex compensation of wavelength dispersion throughout the optical system is obviated. Refraction can occur only at the exit of the cone of rays from the block. However, if the difference of refractive index between the materials in this region can be kept small there should be an advantage over other optical arrangements that leave an air gap between objective and sample and hence have a greater difference in refractive index.

As the present embodiment eliminates such as air space, special optic for launching from air into a curved higher index are not required.

There is merit in incorporating a lubricant between the optical block 20 and the outer sleeve 14, which would assist both optical and mechanical performance, by matching refractive index and reducing friction. Care, must be taken in the choice of the lubricant, however. Some lubricants have compounds with long molecules and become birefringent with shear. This is undesirable in the optical path.

It will also be noted that the cone of rays emerging from the fibre endface does not diverge to the extent of a fibre in air, as occurs with vibrating fibre scanners. This reduces the area of the reflecting surface which is required for a given numerical aperture, as compared to an air path to the reflector.

The use of aspheric optics suggests the use of polymer material for the block, which may reduce the cost to such an extent that the fibre and rotating tube system can reasonably be made disposable. By contrast a major concern with conventional in-line endoscopes using refractive optics is that, although the vibrating scanner can be made very simple and inexpensive, the multi-element optics required between the fibre tip and the focal point remain complicated and relatively expensive; they must be complex to achieve the necessary dispersion and aberration performances. The small diameter of the rotating tube of the present embodiment makes the contrast even more striking.

The optical block 20 can be manufactured by any suitable technique, such as in-mould polymerisation, compression or injection moulding.

Handling of such small optical components as the optical block 20 warrants special attention. The optically sensitive areas are the point 30 at which the fibre 18 is attached to the block 20, the reflective zone on internal surface 26, and the light exit region of block 20 and sleeve 14. The rest of the cylindrical outer surface of sleeve 14 can therefore be safely handled and need not be optically smooth. This consideration is important for polymer components where the soft material is susceptible to surface damage.

Optical Geometry

At the distal end of a flexible endoscope there is generally a rigid section that houses the scanner mechanism and launch optics. The length of this rigid section can be a barrier to navigating sharp bends. For vibrating fibre scanners the length of the scanning mechanism itself can be over 30 mm to which must be added the length of the refractive optics. A 5 mm diameter forward looking endoscope may well have a rigid length over 40 mm. For the rotating tube scanning head 10, there is the length of the optical block and any additional length for a practical connection with the inner driving tube.

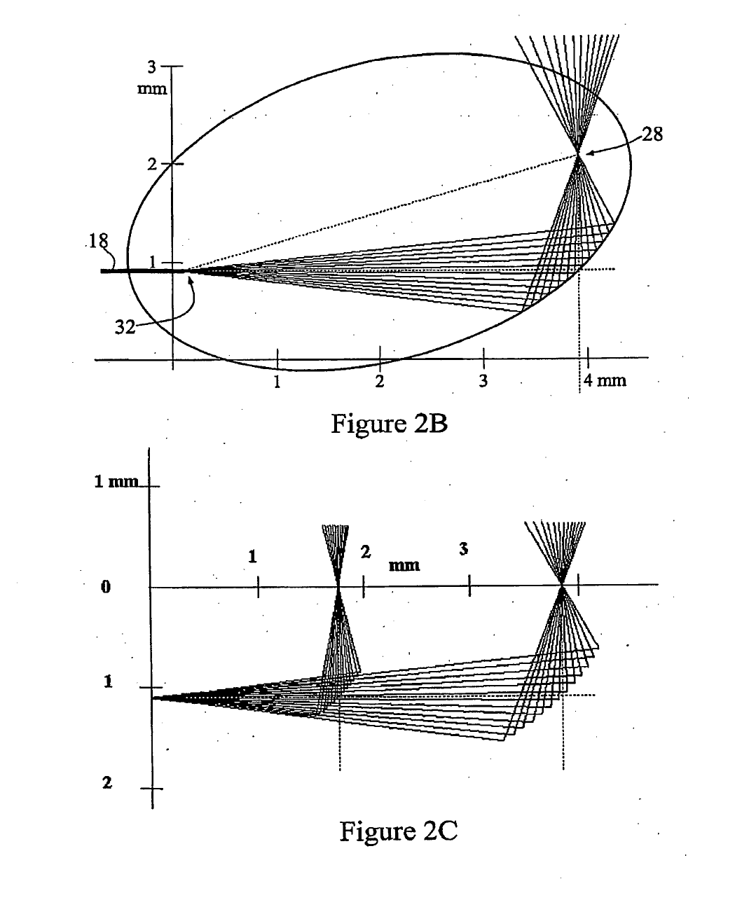

Firstly, the optics of the head 10 are illustrated schematically in FIG. 2A. It will be noted that the internal surface 26 is a portion of section of an ellipsoid with a first focal point at the exit tip 32 of fibre 18 and a second focal point at the point observational field 28. This is also depicted as a schematic plot in FIG. 2B.

FIG. 2C shows illustrates the trade-off between block length and numerical aperture. For a distance between foci (28, 32) of 4 mm, the rigid length is around 4 to 5 mm. As can be seen from the included angle in the exit bundle of rays, the numerical aperture has been increased considerably compared to that of the fibre 18. Leaving the offset at 1.1 mm and reducing the distance between foci to 2 mm, it can be seen that the block length can be reduced, but at the expense of the numerical aperture at the sample (or focus 28). The maximum numerical aperture is limited by the rays from the fibre diverging beyond the radial dimensions of the block 20 before reaching the reflecting internal surface 26. In practice, before this limit is reached the asymmetry in the exit rays may present a problem. It should be noted that, even in the illustrated example, some asymmetry is apparent in the rays of the exit bundle for the 4 mm case. Even so it can be concluded that the side viewing optics can be made shorter by an order of magnitude when compared with a scanner and refractive optics of the forward looking endoscope.

Confocal Operation

Optical sectioning involves manipulating the focal point to some controlled depth below the surface of the sample. For the side viewing scanner some means would be useful for adjusting the radial distance of the focus beyond the outer surface of the external fixed sleeve 14. Since the optical system between fibre tip and focal point is fixed in this embodiment, some other method is must be used to vary the optical distance between the exit surface of the block 20 and the outer surface of the external sleeve 14.

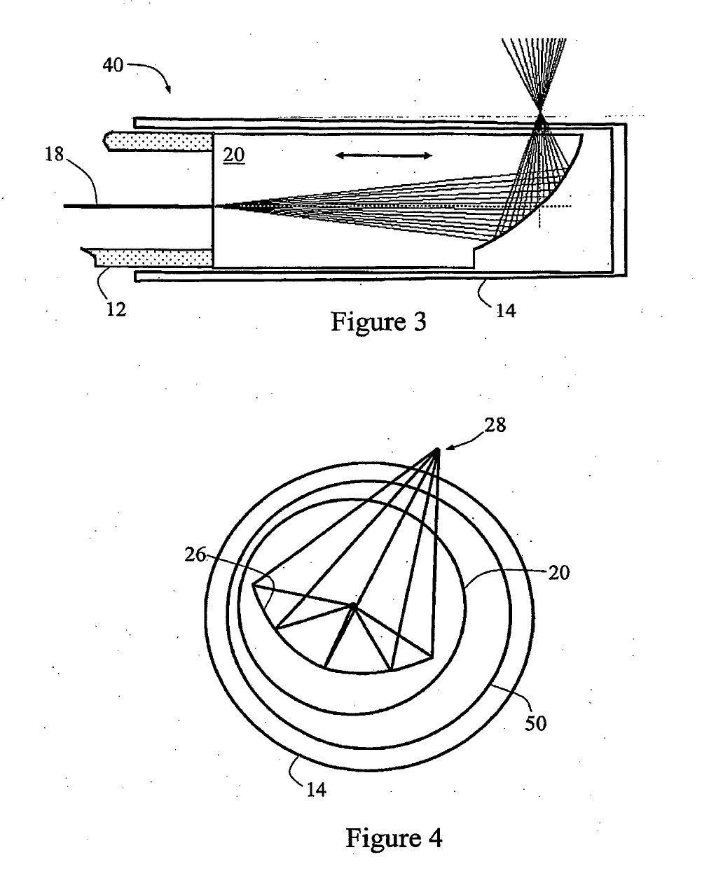

One such technique is shown in FIG. 3, which does not require additional components. In this embodiment, the wall thickness of the external sleeve is non-uniform, varying from about 400 micron at the forward (or fibre) end of the head 40 down to 100 micron at the rear end. Then moving the inner tube 12 and optical block 20 combination longitudinally relative to the outer sleeve 14 varies the penetration of the point observational field beyond the outer surface of sleeve 14 over a 292 micron range. Provided the sample is pressed against the sleeve 14, as would be expected in a blood vessel, the point observational field can be varied in depth in the sample.

The longitudinal movement of the inner tube 12 thus provides the depth scan and is not available for scanning in any other direction.

The taper needed for the depth scan could alternatively be introduced circumferentially by using an eccentric transparent rotatable polymer sleeve 50 located between the block 20 and the fixed outer sleeve 14, as shown schematically in FIG. 4. As the rotatable sleeve 50 is rotated relative to the block 20, the depth beyond the rotatable sleeve 50 and therefore outer sleeve 14 to which the light is focussed is varied. An additional mechanism such as a third tube attached to the sleeve and rotating with the tube would be used for driving the optical block 20. In this embodiment, it is envisaged that a refractive index matching fluid may be desirable between the block 20 and rotatable sleeve 50, and between the rotatable sleeve 50 and the outer sleeve 14.

In some variations of this embodiment, it may be sufficient to provide the outer sleeve 14 itself with a circumferentially non-uniform thickness, but such embodiments would typically require independent control of the rotation of the block 20 and the sleeve 14 so that the view can be rotated and depth varied independently.

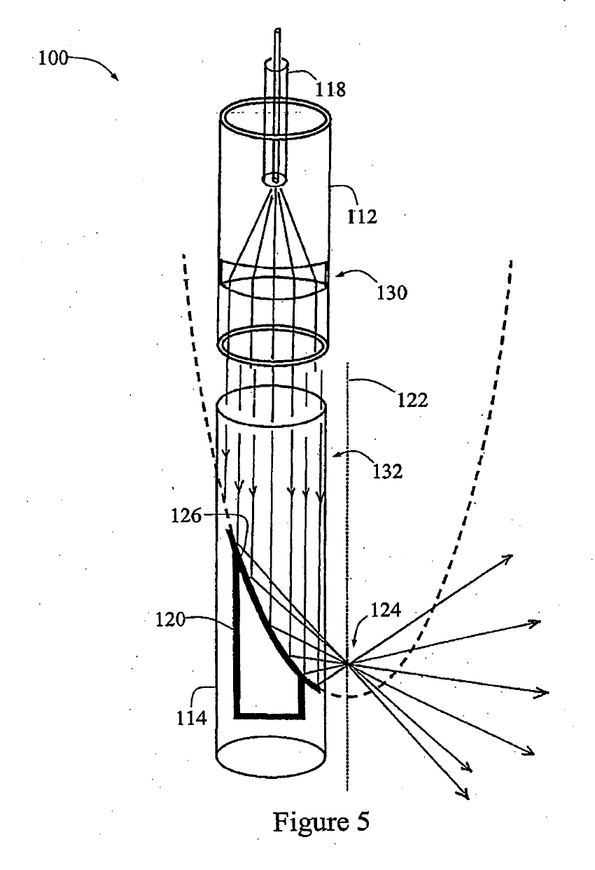

In a still further embodiment, a scanning device for use as a confocal probe for the oesophagus (for example) is provided. In this embodiment, shown schematically in FIG. 5, employs focussing elements located optically after the light emerges from the fibre tip so that the divergent beam is transformed into a parallel beam. A paraboloid rather than ellipsoid mirror is then employed to direct the light laterally and focus the parallel beam to a point observational field.

Thus, referring to FIG. 5, the scanning device 100 includes an inner tube 112 that accommodates the fibre 118 for transmitting light to the scanning device 100. As with head 10 of FIG. 1, the device 100 includes a transparent outer tube 114, which is fixed and houses an off-axis paraboloid focussing mirror 120 (with axis of symmetry 122). In addition, the device 100 includes collimating elements in the form of an achromatic collimating lens 130 for focussing the light diverging from the tip of fibre 118 into a parallel beam 132. The advantage of the parallel beam is that the location of the focussing mirror 120 can be varied along tube 114 without affecting the manner in which the light impinges on the focussing mirror 120. The benefits of this are discussed below. In all cases, therefore, the parallel beam 132 is focussed by mirror 120 to a point 124 outside tube 114.

The paraboloid of revolution is the limiting case of the ellipsoid, and has the property that it takes a beam of parallel light rays (which are parallel to the axis of revolution) and brings them to a point focus 124. This focus, as has been seen, can be outside the tube 114 as with the embodiments the ellipsoid reflective surfaces.

Focussing mirror 120 is formed as a separate integer (with the mirror reflective surface operation either in air or with the rays reflected back into the optical polymer). Consequently, it can be rotated for scanning, as in the embodiments described above, effected with moving the, fibre 118 or the collimating optics 130. The beam 132 is parallel, so focussing mirror 120 can be translated along tube 114 to provide longitudinal scanning so that a scanned image can be formed. Suitable means of effecting this translation are described below.

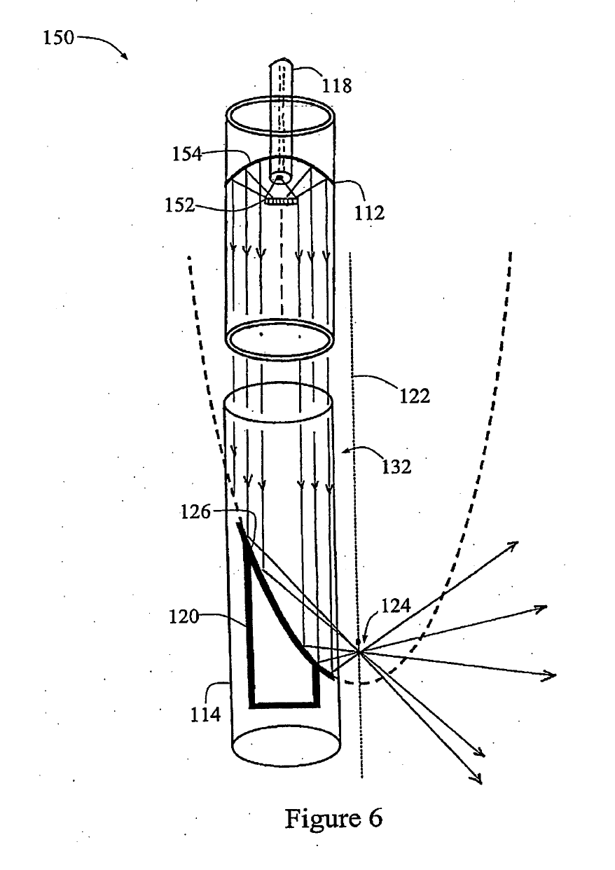

FIG. 6 illustrates a variation 150 of the device 100, in which—instead of an achromatic lens—a pair of on-axis mirrors 152,154 are used to collimate the beam emerging from the fibre 118. This device 150 is in other respects the same as the previous device 100.

Thus, a planar mirror 152 is located in front of the exit tip of the fibre 118 and perpendicular to the direction of the emerging beam. This mirror reflects the beam back towards the fibre 118. A second mirror 154, of parabolic shape, is located about the fibre 118 (having an aperture to accommodate the fibre 118). This parabolic mirror 154 collimates the beam and reflects back along the device 150.

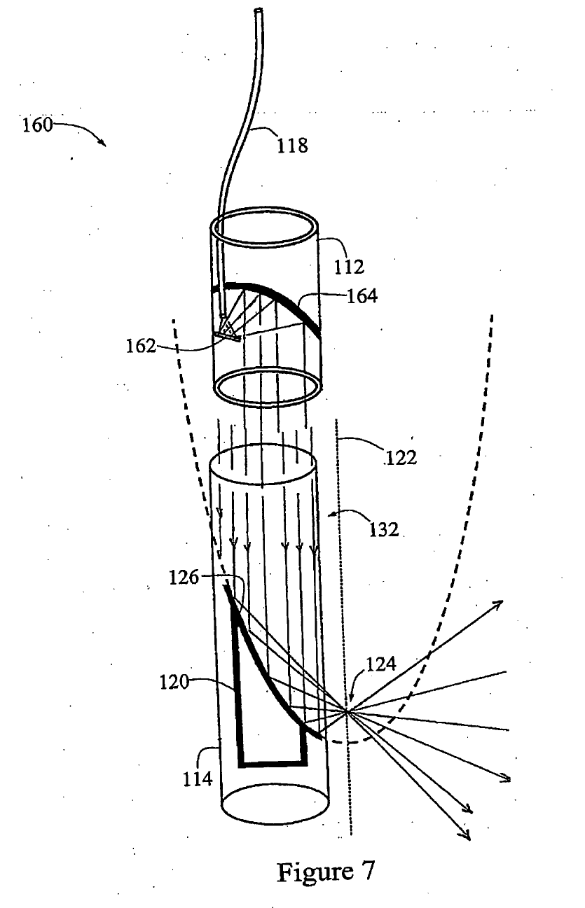

FIG. 7 illustrates an off-axis variation 160 of this use of collimating mirrors, in which the fibre 118 is off-axis and a pair of off-axis mirrors 162,164 are used to collimate the beam emerging from the fibre 118. This device 160 is in other respects the same as the previous device 150. It will be noted that the mirrors are off-axis with respect to the tube 114, but that the parabolic mirror 164 has an axis of symmetry aligned with the fibre 118

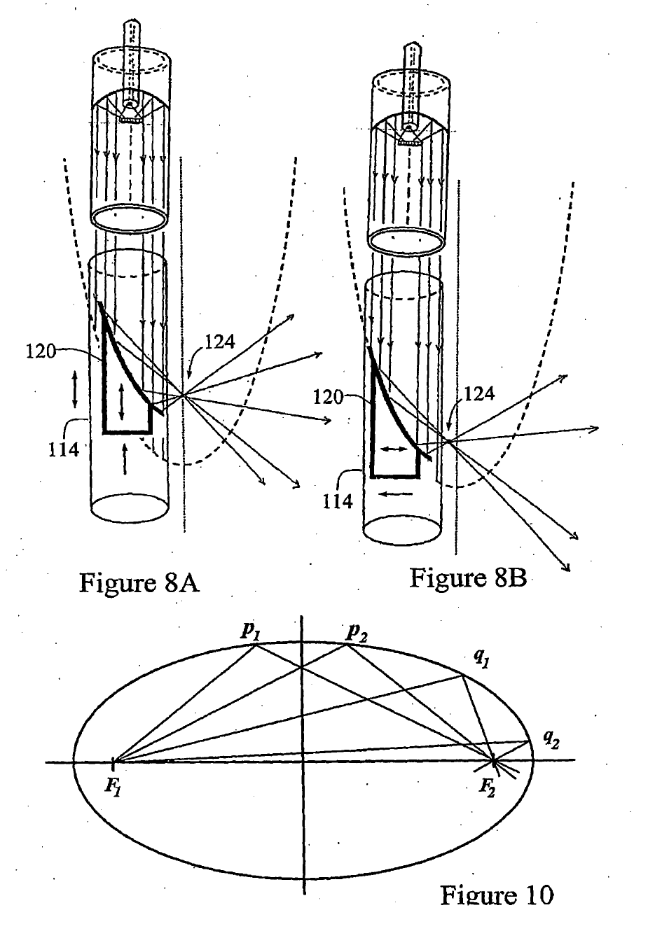

FIGS. 8A and 8B illustrate scanning by means of the paraboloid mirror 120 of the embodiment of FIG. 6. Scanning is effected in like manner in the embodiments of FIGS. 5 and 7, or other electromagnetic, pneumatic, hydraulic or other actuation mechanisms can be employed.

The initial position of the paraboloid mirror 120 is shown in FIG. 6. FIG. 8A illustrates the motions of the mirror for axial or longitudinal scanning, which can be effected by any suitable means, such as a stepper motor or a pneumatic drive located behind the mirror 120. As the mirror 120 is advanced or retracted within and relative to tube 114, the point observational field 124 moves in parallel with the mirror 120 so that a longitudinal scan is performed.

Other methods for effecting longitudinal scanning are possible. For example, a micro-grooved thread may be turned on a section of metal on the outside of the rotating mirror 120. This thread can be produced with a fine diamond point and it can work as a screw thread with a pitch of less than a micron. The matching thread can be formed by pressing the metal surface against clamping sections of soft plastic, such as Teflon tape.

The rotation of the mirror would thus also serve to advance the assembly by one micron (or whatever pitch is used) per revolution thus generating a raster.

It would also be possible to produce a fast axial or longitudinal scanning motion of the focussed spot by reciprocating vibration mirror 120. Such an oscillatory motion could be produced electro-magnetically. A refinement of this design employs a counterweight that vibrates 180° out of phase to cancel unbalanced forces.

Resonant oscillatory rotational scanning movement of the mirror could also be achieved by a similar arrangement.

FIG. 8B illustrates the motions of the mirror for depth scanning, that is, for focussing at greater or lesser depth within the sample (such as tissue). As the mirror 120 is moved laterally within and relative to tube 114, the point observational field 124 moves towards or away from the tube 114, thus scanning to less or greater depths.

This motion can also be effected by any suitable means, such as an electromagnetic or pneumatic drive (such as an inflatable sac) located between the mirror 120 and the tube 114.

In all of the above embodiments, in order to provide 360° circumferential scanning a miniature electric motor and gearbox (not shown) can be employed to rotate the mirror. In the paraboloid mirror embodiments, the motor may be mounted on a piston that can be advanced in an axial or longitudinal direction by the movement of fluid down a pipe into the piston. This arrangement provides, therefore, simultaneous scanning in two orthogonal directions (viz. longitudinal and rotational).

In some of the above embodiments, astigmatism is produced by the difference in radius of cylindrical curvature between the inner and outer surfaces through which light passes. In FIG. 1, these surfaces are the outer surface of the block 20, and the sleeve 14. These will not a great difference in radius of curvature, but it will be appreciated that, in the embodiments shown in FIGS. 5 to 7, this difference will be considerably greater.

Further, fluid or tissue in contact with either or both of the surfaces will change the degree of astigmatism in the system.

The optical systems disclosed in FIGS. 1 and 3 will have essentially no astigmatism if the plastic of block 20, the sleeve 14 and any lubrication liquid between them have refractive indices close to the refractive index of the sample. For human tissue this is approximately that of water, or n≈1.33. Indeed these systems should have essentially no optical aberrations when imaging point to point. There are available optically clear polymer materials with refractive indices close to that of tissue and water.

In the event that it is difficult to mass-produce these shapes with such low refractive index materials there are a number of possible alternative ways to achieve that same result.

Firstly, in the embodiments of FIGS. 5 to 7, the mirror 120 may be rotated in a fluid within the transparent tube 114. The refractive index of the liquid should be 1.33, approximating the refractive index of the tissue to be examined. If the material of which the tube 114 is made is of a substantially higher refractive index then the liquid should also be of a refractive index higher than 1.33 by an amount dependent on the refractive index of the wall material of tube 114 and its thickness.

In the embodiments of FIGS. 1, 3 and 4, it would also be possible to include a moulded cylindrical surface, at the surface where the light exist the optical block 20 in the direction of the specimen or sample.

Secondly, astigmatism can be compensated for by providing additional aspherical elements in the optical path. Astigmatism in the embodiments of FIGS. 5 to 7 can be compensated for by means of cylindrical optical elements that rotate with the focussing mirror 120, and placed just inside the viewing section (i.e. through which the sample is viewed) of the outer tube 114.

Examples of suitable cylindrical elements are shown in FIGS. 9A and 9B, in which cylindrical elements 172,174 are adapted to be located inside tube 114. Referring to FIG. 9A, correcting element 172 has a planar incident surface 176 and a cylindrical exit surface 178. The cylindrical element 174 of FIG. 9B has a cylindrically concave incident surface 180 and a cylindrical exit surface 182.

Some polymers that might be used to manufacture outer tube 114 may, in the sleeve manufacturing process, become birefringent. If this is the case, it will be necessary to put a waveplate of similar strength into the beam path, with its fast axis oriented at right angles to the fast axis of the tube wall. A small piece of the tube wall could be cut out and used for this purpose.

Cylindrical lenses can, alternatively, be introduced at an earlier point in the optical path. It would also be possible to figure the mould that produces the surface of the focussing mirror 120 so as to include an optically cylindrical component.

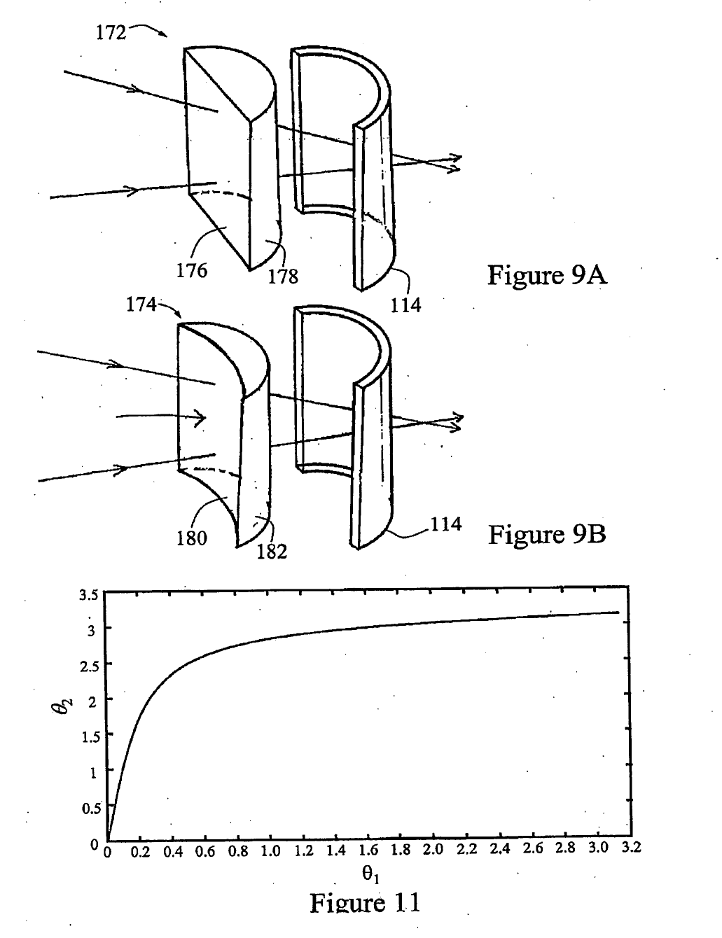

It may also be possible, in some embodiments, to provide variable magnification by controlling fibre orientation. FIG. 10 shows elliptical mirror with eccentricity 0.84 focussing the field from a fibre tip at the focus F1 onto focus F2. With the fibre oriented so that it casts lightcone F₁ p₁ p₂ onto the mirror, lightcone p₁ p₂ F₂, which is the mirror image of lightcone F₁ p₁ p₂ reflected in the ellipse's minor axis, is brought to a focus at F₂ such that the numerical aperture of the cone converging on the object is precisely equal to that of the cone launched from the fibre. The magnification of this imaging system is then precisely unity and small movements in the fibre tip are translated into motions of the same magnitude and opposite sense in the tip's image.

By contrast, if the fibre is now tilted, so that its tip stays at the focus F₁ but casts lightcone F₁ q₁ q₂ onto the mirror, the lightcone q₁ q₂ F₂ is now of much higher numerical aperture that that of the cone launched from the fibre. Small tip movements are now translated into magnified movements of the tip's image.

The angle θ₁ between a ray launched from F₁ and the major axis is related to the angle θ₂ between the projected ray through focus F₂ and the major axis by:

$$sin ( {θ}_{2} ) = \frac{( 1 - {ɛ}^{2} ) sin ( {θ}_{1} )}{1 + {ɛ}^{2} - 2 ɛ cos ( {θ}_{1} )} ,$$

where ε is elliptical eccentricity. The relationship between θ₁ and θ₂, is plotted in FIG. 11, with θ₁ in radians plotted along the horizontal axis and θ₂ in radians along the vertical axis, for an eccentricity of 0.84. The maximum magnification of the device is given by the gradient of this curve at θ₁=0 and is:

$${M}_{max} = - \frac{1 + ɛ}{1 - ɛ} .$$

More generally, the magnification for a small numerical aperture lightcone is given by:

$$M = - \frac{( 1 - {ɛ}^{2} ) cos ( θ ) + 2 ɛ}{{( 1 + {ɛ}^{2} - 2 ɛ cos ( θ ) )}^{2}} ( 1 - {ɛ}^{2} ) ,$$

where θ is the angle between the chief ray of the lightcone launched from the fibre and the major axis.

This ability to vary the NA of the focussed beam is likely to be of considerable particular value in those embodiments of the present invention adapted for optical coherence tomography.

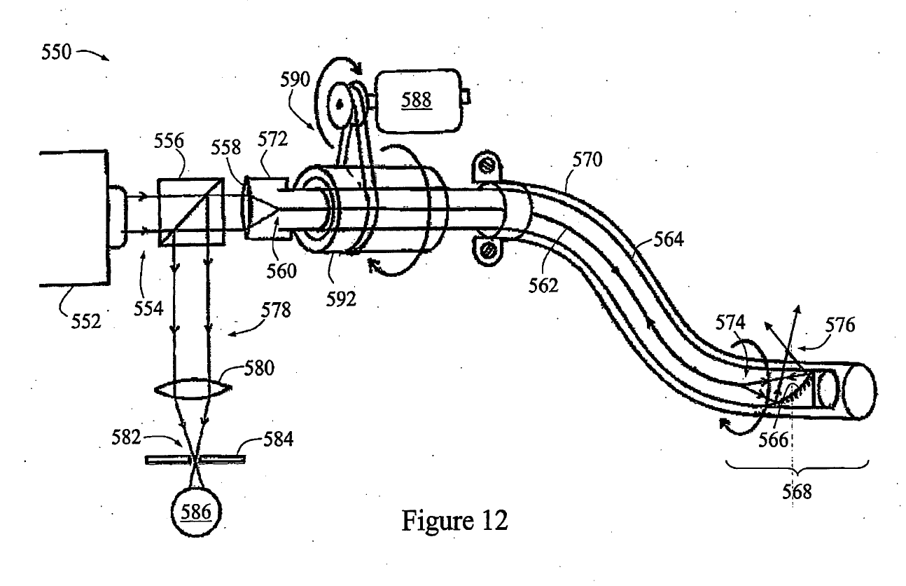

A scanning confocal endomicroscope according to another embodiment of the present invention is shown generally at 550 in FIG. 12. Endomicroscope 550 includes a laser source 552 for providing a light beam 554, a beamsplitter 556 and a lens 558 for receiving the beam 554 and focussing it into the proximal end 560 of the core of an optic fibre 562. The fibre 562 is enclosed in a rotatable tube 564 coupled to a ellipsoidal mirror 566 (located in the optical head 568 of the endomicroscope 550), for rotating the mirror. The tube 562 is, in turn, houses in a transparent sleeve 570. The lens 558 and proximal end 560 of the fibre 562 are mounted in a cylindrical housing 572, which is also coupled to and rotates with the tube 564.

If endomicroscope 550 is configured to operate as a multi-photon endomicroscope, laser source 552 would be a pulsed laser source.

The beam 554 exits the distal tip 574 of fibre 562 and is reflected by mirror 566, through the sleeve 570, and focussed by ellipsoidal mirror 566 to a point observational field 576 in or on the specimen (e.g. a tissue sample).

Some of the light re-emitted from the point observational field 576 is returned by the mirror 566 back into the optic fibre 562, and travels to the beamsplitter 556 which reflects it—or a portion thereof—laterally as beam 578. This beam 578 is focussed by lens 580 through an aperture 582 in a spatial filter 584 and impinges on a photodetector 586.

The system also includes an electrical motor 588 that, by means of a belt drive 590, in use rotates a drum 592. The drum 592 encloses a portion of the tube 564, and hence rotates tube 564 and, ultimately, optic fibre 562 and mirror 566, so that scanning is effected.

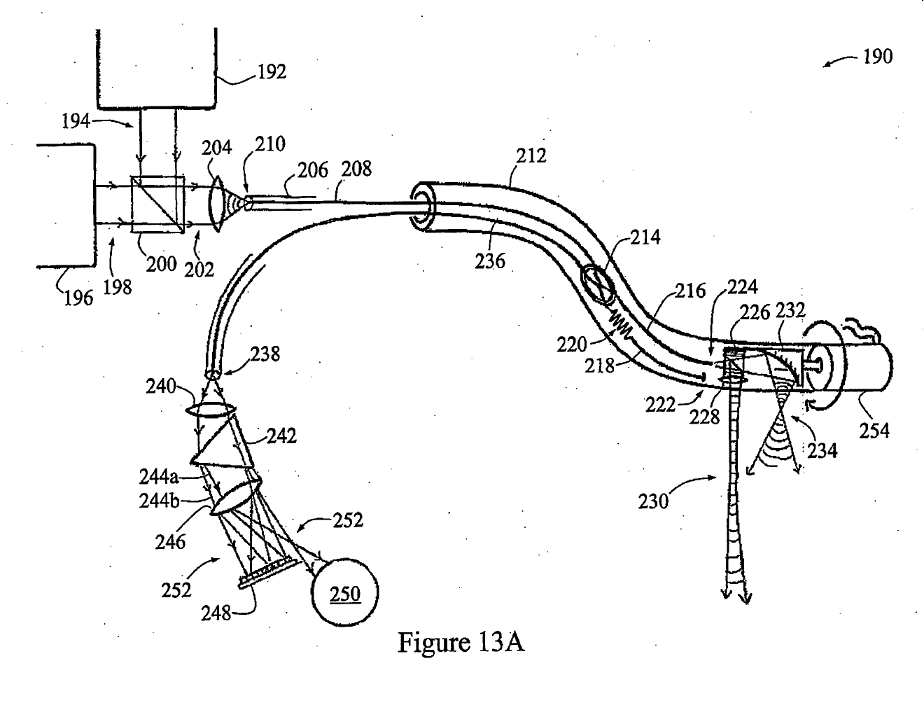

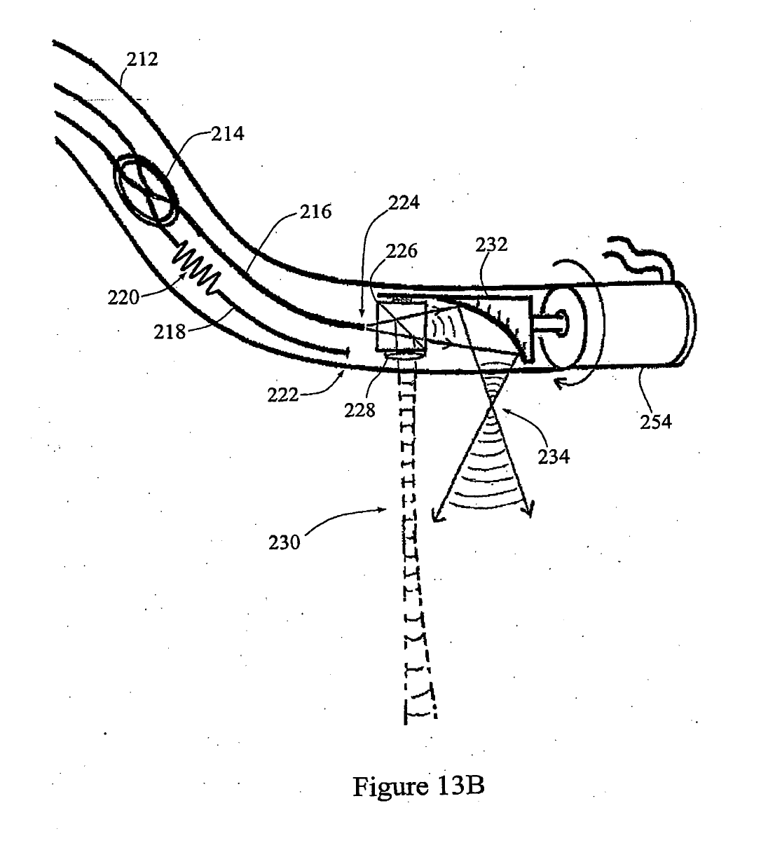

FIG. 13A is a schematic view of a scanning confocal endomicroscope and optical coherence tomography (OCT) imaging system 190 according to another embodiment of the present invention. The confocal fluorescence and the OCT uses the same fibre, fused biconical tapered coupler (FBTC), mirror for directing light into the sample and scanning motor for rotating that mirror. FIG. 13B is a more detailed view of the optical head region of the system 190.

Thus, referring to FIGS. 13A and 13B, the scanning confocal endomicroscope and OCT imaging system 190 includes a blue laser 192 for providing a light beam 194, a incoherent infrared source 196 for providing a low coherence length infrared beam 198, and a first dichroic beamsplitter cube 200 for combining the beams 194 and 198 into a combined beam 202.

The system 190 includes a lens 204 for receiving and focussing the combined beam 202, and an optic fibre 206 for transmitting the combined beam 202 to the optical head (described below) of the system 190. The lens 204 is positioned so as to direct the combined beam 202 into the core 208 of the optic fibre 206, at the tip 210 of the optic fibre 206.

The system 190 includes a transparent sheath 212 that houses the optic fibre 206 for the bulk of the fibre's length, and a fused biconical tapered coupler (PIM) 214 housed within the sheath 212. When the combined beam 202 reaches the coupler 214, the coupler divides the beam between first and second output legs 216 and 218. Second leg 218 has extra length 220 to allow it to act as an interferometer path (with light passing into second leg 218 being reflected from its terminus 222) by matching the path length of light that is transmitted to the specimen.

Light directed into first leg 216 reaches and exits the distal tip 224 of the first leg. The system 190 includes, within the sheath 212, a second dichroic beamsplitter cube 226, located to receive light exiting distal tip 224, and a lens 228 located to receive and focus light reflected perpendicularly by the second beamsplitter cube. Thus, second beamsplitter cube 226 reflects the infrared component of the light emerging from distal tip 224 towards lens 228, which focuses the infrared component through the wall of sheath 212 to an elongated focal volume 230 within the specimen. The blue light component of that portion of the combined beam that is launched into first leg 216 is for confocal fluorescence imaging, and also exits distal tip 224 but passes through the second beamsplitter cube 226 and is reflected by a mirror 232 of ellipsoidal figure (also provided within sheath 212). The mirror 232 reflects the diverging light beam laterally and focuses it to a high NA point observational field 234.

Return light from elongated focal volume 230 and return light from point observational field 234 retrace their respective paths back into first leg 216 and to the coupler 214. A portion of this light is directed into third fibre leg 236 and passes to the tip 238 of that leg, from which it exits.

The system 190 includes a collimating lens 240 located to collimate light exiting the tip 238 of third fibre leg 236, a prism 242 for then receiving that light and for separating it into its spectral components (indicated schematically at 244a, 244b), and a further lens 246 for focussing these spectral components. The system 190 includes a linear CCD array 248 and a photomultiplier tube 250. The CCD array 248 is located to collect the infra-red spectrum 252 (to provide the information for the optical coherence tomography image), the photomultiplier tube 250 is located to collect the (visible) fluorescence component 252, to provide the information to produce a confocal fluorescence image. It will be appreciated that the fluorescence component 252 will usually also include some reflected light.

In this embodiment, the system 190 includes an electrical motor 254 located at the optical head 256 and hence adjacent to the mirror 232. The motor can be controlled by known means to rotate the mirror 232, so that the mirror scans the incident light around the specimen. However, as in previously described embodiments (such as that of FIG. 12), this embodiment could alternatively employ a rotating tube located within the sheath 212 and coupled to the mirror 232 to provide this function.

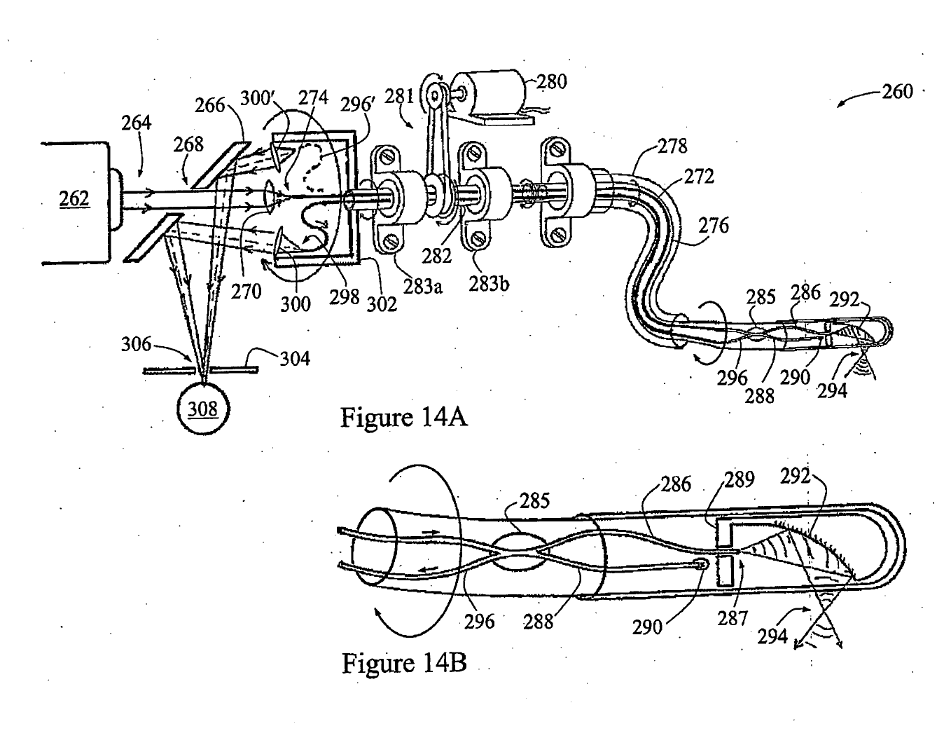

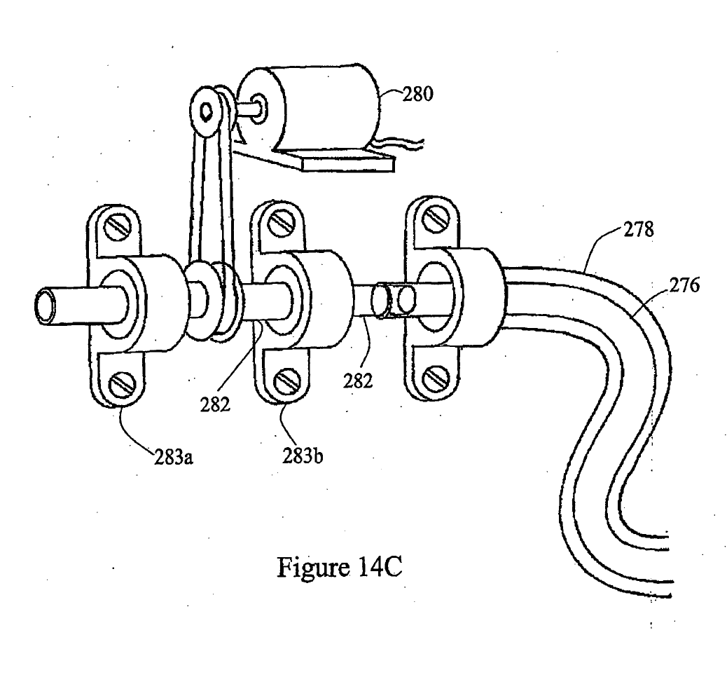

FIG. 14A is a schematic view of a scanning confocal endomicroscope system 260 according to another embodiment of the invention. In the embodiment of FIG. 14, the beamsplitter is a fused biconical taper coupler (FBTC) located close to the distal tip of the endomicroscope. This arrangement reduces optical noise but requires a separate return pathway that can also couple light to a stationary photodetector while the mirror in the endoscope head is rotating. FIG. 14B is a more detailed view of the optical head region of the system 260, and FIG. 14C is a more detailed view of the region of the motor and belt drive of the system 260.

Thus, referring to FIGS. 14A, 14B and 14C, system 260 includes a laser 262 for launching a light beam 264, a mirror 266 (arranged obliquely to the beam) that has an aperture 268 for admitting the beam 264, a lens 270 located to then receive and focus the beam 264, and an optic fibre 272 with a proximal tip 274 into which the lens 270 couples the beam 264. As will be appreciated by those skilled in the art, the lens 270 and proximal tip 274 of optic fibre 272 are positioned to be fixed relative to each other.

The system 260 includes a flexible tube 276 that houses the optic fibre 272 over the bulk of the fibre's length, but the principal role of the tube 276 is to rotate the system's mirror 292, as will be discussed below. In addition, the system 260 includes a flexible transparent sleeve 278 that houses the flexible tube 276 and hence optic fibre 272. The system also includes an electrical motor 280 that, by means of a belt drive 281, in use rotates a rigid tube 282 (rotatably mounted between a pair of brackets 283a, 283b) whose proximal end is coupled to flexible tube 276. Tube 276 is thus rotated by the rigid tube 282 within sleeve 278, and tube 276 in turn rotates optical fibre 272 and mirror 292.

The system includes an FBTC 284 to which the fibre 272 is coupled (within and hence rotated with tube 276), and which has first and second output fibre legs 286 and 288. Consequently, light launched into optic fibre 272 is split into two portions, a first portion launched into first output fibre leg 286 and a second portion launched into second output fibre leg 288. Second fibre leg 288 is terminated by an index matching AR device 290.

First fibre leg 286 has an exit tip 287 that is held in a disc-like mount 289, and emits light towards ellipsoidal mirror 292; mount 289 is, in this embodiment, integral with the mirror 292. Mirror 292 reflects the light through the transparent wall of the sleeve 278 and focuses it to a point observational field 294 on or within a specimen (such a biological tissue).

Light re-emitted from this observational field 294 towards ellipsoidal mirror 292 is reflected by the mirror and coupled back into the first fibre leg 286, travelling back in the opposite direction.

On reaching the FBTC 284 the return light is split, a portion travelling along third fibre leg 296 located in the rotating tube and thence to the exit tip 298 of the third fibre leg 296. A lens 300 is provided at the exit tip to receive and condense light emerging from that tip. The lens 270, distal tip 274 of optic fibre 272, exit tip 298 of the third fibre leg 296 and lens 300 are all located in a otherwise hollow cylinder 302 coupled to the rotating rigid tube 282, open at its proximal end and generally closed (apart from admitting rigid tube 282) at its distal end; hence these elements and cylinder 302 rotate with the rigid tube 282 and the flexible tube 276. Indeed, lens 270, distal tip 274 of optic fibre 272, exit tip 298 of the third fibre leg 296 and lens 300 are mounted ultimately to the cylinder 302 so that their relative positions are fixed.

The light is projected by lens 300 onto the mirror 266, which reflects the light generally laterally. The system 260 includes a spatial filter 304 with an aperture 306, and a photodetector 308. The laterally projected light is directed through aperture 306 and thence onto photodetector 308, which generates an electrical signal that can then be reconstructed into an image by known techniques.

It can be seen that when the endoscope has rotated by 180° that the beam from the third fibre leg 296 and lens 300 are rotated into positions 296′ and 300′ respectively, but that light emitted from the third fibre leg will nonetheless also be projected onto the photomultiplier tube. The same is true for intermediate positions (not shown).

It may be desirable, in some applications to completely separate the outgoing and returning light paths and to avoid putting an FETC in the rotating portion of the endomicroscope. This can be achieved according to the present invention by means of a paraboloidal mirror and polarisation separating beamsplitter, most conveniently by delivering plane polarised light at the tip.

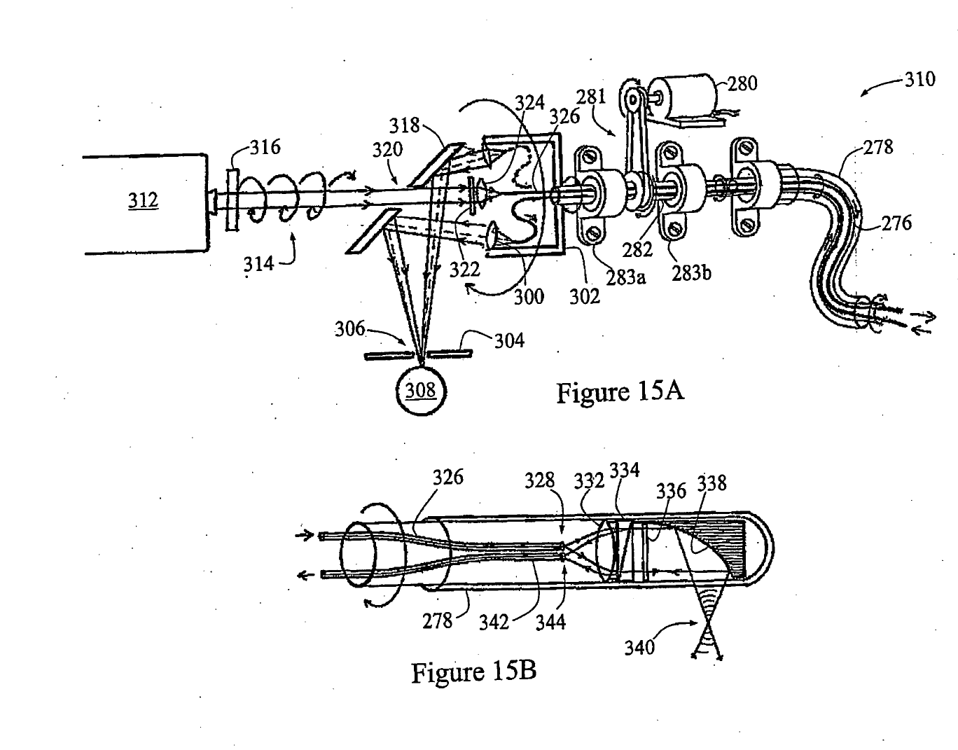

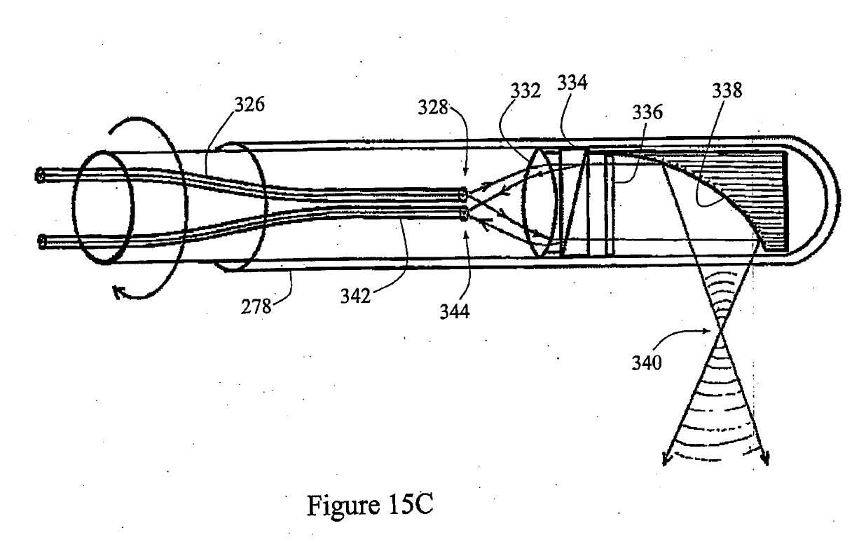

FIGS. 15A and 15B are schematic views of first and second portions respectively of a scanning confocal endomicroscope 310 according to another embodiment of the present invention; FIG. 15B is of a somewhat larger scale than is FIG. 15B. FIG. 15C is a further enlarged version of FIG. 15B, corresponding to the optical head region of endomicroscope 310. Endomicroscope 310 employs rotating components comparable to those of the embodiment shown in FIGS. 14A, 14B and 14C.

Endomicroscope 310 includes a laser source 312 that provides a beam of plane polarised light 314, and a quarter wave plate 316 set at 45° that receives and circularly polarises the light 314. The endomicroscope 310 includes a mirror 318 (arranged obliquely to the light 314) that has an aperture 320 for admitting the light 314, a further quarter wave plate 322, a lens 324 and a polarisation maintaining fibre 326. The further quarter wave plate 322, lens 324 and fibre 326 are mounted in fixed relationship and to rotate together, driven—as will be described below—in a manner comparable to that of the embodiment of FIGS. 14A, 14b and 14C.

Lens 324 is located to receive and focus the light 314 and then couple the light 314 into fibre 326.

The endomicroscope 310 includes a rotating flexible tube and drive system comparable to that of FIGS. 14A, 14B and 14C, to which the reader should refer for details; like reference, numerals have been used to indicate like features.

However, unlike the system of FIGS. 14A, 14B and 14C, endoscope 310 does not include an FBTC. Rather, light 314 traverses the entire length of fibre 326 and, on reaching the distal tip 328 of the fibre 326, the light is emitted as a divergent beam 330 that is collimated by a lens 332, passes through a Nomarski type prism 334 and then passes through a broadband quarter waver plate 336 (or other waveplate) to a paraboloidal mirror 338. The mirror 338 reflects the light laterally through the transparent wall of sleeve 278, and focusses the light to a point observational field 340.

The return light from the point observational field 340 is largely diverted by the Nomarski prism 334 to enter the core of a return fibre 342, whose entry tip 344 is adjacent to but displaced from exit tip 328 of fibre 326. The return light then travels to the other (or exit) end 346 of return fibre 342, from which it emerges and is projected by lens 300 via mirror 318 through an aperture 306 in spatial filter 304 onto photodetector 308.

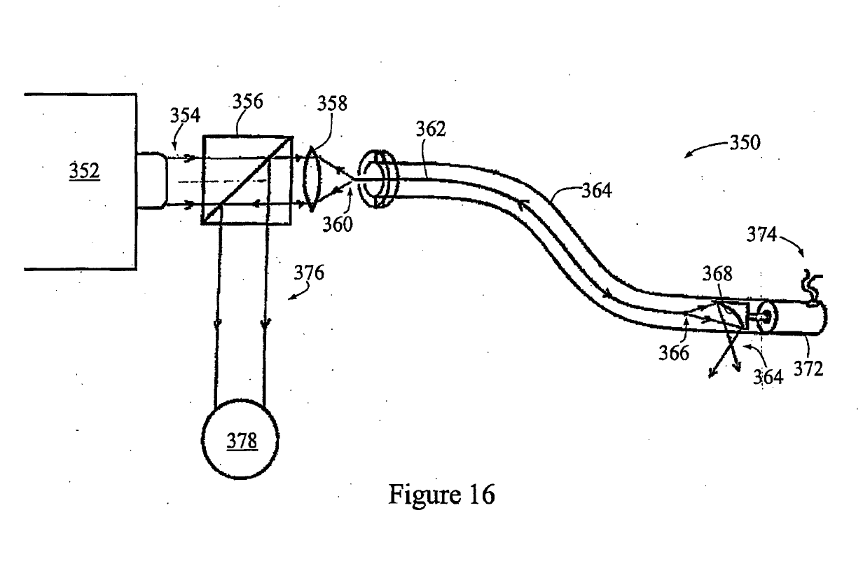

A scanning confocal endomicroscope according to another embodiment of the present invention is shown generally at 350 in FIG. 16.

Endomicroscope 350 includes a laser source 352 for, providing a light beam 354, a beamsplitter 356 and a lens 358 for receiving the beam 354 and focussing it into the proximal end 360 of the core of an optic fibre 362. The fibre 362 is enclosed in a transparent sleeve or jacket 364.

The beam 354 exits the distal tip 366 of fibre 362 and is reflected by an ellipsoidal mirror 368, through the jacket 364, and focussed by ellipsoidal mirror 368 to a point observational field 370 in or on the specimen (e.g. a tissue sample). The endoscope 350 includes, on the remote side of mirror 368 and also within jacket 364, an electrical motor 372 for rotating the mirror 368 in order to effect scanning. As in other comparable embodiments, power and control signals for driving and controlling the motor 372 are provided to the motor along wires 374 that are run along the outside of the jacket 364 or—if preferred—fed along the interior wall of the jacket.

Some of the light re-emitted from the point observational field 340 is returned by the mirror 368 back into the optic fibre 362, and travels to the beamsplitter 356 which reflects it—or a portion thereof—laterally as beam 376. This beam 376 impinges on a photodetector 378.

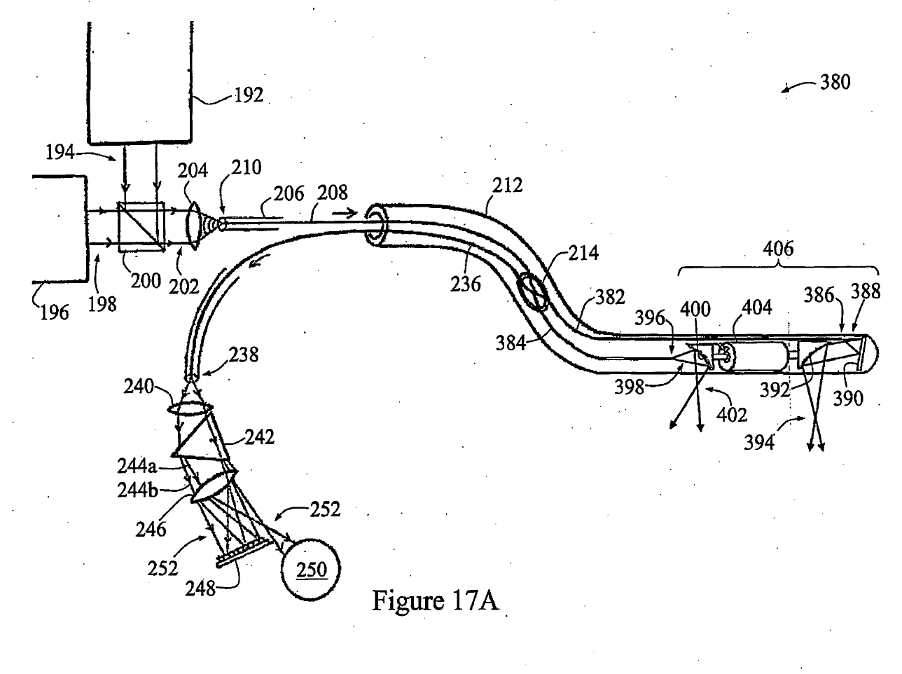

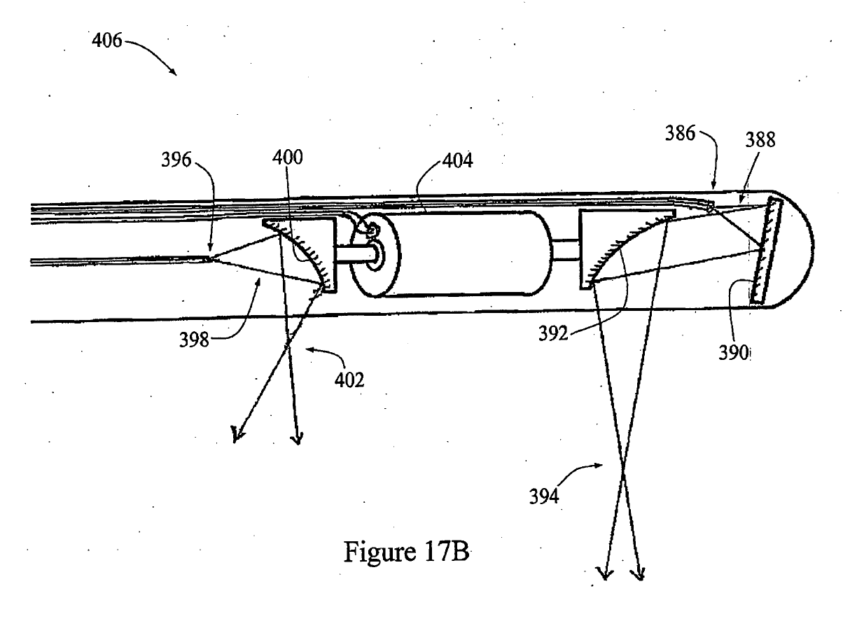

FIG. 17A is a schematic view of a scanning confocal endomicroscope and OCT imaging system 380 according to another embodiment of the present invention, comparable in many respects to system 190 of FIGS. 13A and 13B. Consequently, where elements of system 380 are essentially identical to like elements of system 190 of FIGS. 13A and 13B, like reference numerals have been used. FIG. 17B is a more detailed view of the region of the optical head 406 of the system 380 of FIG. 17A.

Referring to FIGS. 17A and 17B, scanning confocal endomicroscope and OCT imaging system 380 includes a blue laser 192 for providing a light beam 194, a incoherent infrared source 196 for providing a low coherence length infrared beam 198, and a dichroic beamsplitter cube 200 for combining the beams 194 and 198 into a combined beam 202.

The system 380 includes a lens 204 for receiving and focussing the combined beam 202, and an optic fibre 206 for transmitting the combined beam 202 to the optical head (described below) of the system 380. The lens 204 is positioned so as to couple combined beam 202 into the core 208 of the optic fibre 206, at the tip 210 of the optic fibre 206. The system 380 also includes a transparent sheath 212 that houses the optic fibre 206 for the bulk of the fibre's length, and an FBTC 214 housed within the sheath 212.

FBTC 214 has first and second output fibre legs 382 and 384. Infra-red light in first fibre leg 382 travels to the distal tip 386 of the first fibre leg and is emitted as a beam 388, which is reflected from a plane mirror 390 onto an ellipsoidal mirror 392 and through the sheath 212. The ellipsoidal mirror 392 focusses the beam 388 to an elongated focal volume 394, such as in a specimen.

Second fibre leg 384—like second fibre leg 218 of system 190 of FIG. 13A—includes extra fibre length (not shown) so that light in both legs traverses the same path length, so that interferometry can be performed. In second fibre leg 384, blue light and infra-red both travel to its distal tip 396. The infra-red light is reflected back by a dichroic layer at the tip (or by Fresnel reflection) to provide a reference beam for interferometry. The blue light is emitted from the distal tip 396 of second fibre leg 384 as beam 398, is reflected by a second ellipsoid mirror 400 through transparent sheath 212 and focussed to a point observational field 402 that, in use, would be in or on a specimen.

Return light from elongated focal volume 394 and return light from point observational field 402 retrace their respective paths back to FBTC 214. A portion of this light is directed into third fibre leg 236 and passes to the tip 238 of that leg, from which it exits.

The system 380 includes a collimating lens 240 located to collimate light exiting the tip 238 of third fibre leg 236, a prism 242 for then receiving that light and for separating it into its spectral components (indicated schematically at 244a, 244b), and a further lens 246 for focussing these spectral components. The system 380 includes a linear CCD array 248 and a photomultiplier tube 250. The CCD array 248 is located to collect the infra-red spectrum 252 (to provide the information for the optical coherence tomography image); the photomultiplier tube 250 is located to collect the (visible) fluorescence component 252, to provide the information to produce a confocal fluorescence image. It will be appreciated that the fluorescence component 252 will usually also include some reflected light.

In this embodiment, the system 380 includes an electrical motor 404 located in the optical head 406, between the ellipsoidal mirrors and hence adjacent to the mirror 232. The motor is controlled by known means to rotate the mirrors 392, 400, so that the mirrors scan the incident light beams around the specimen. However, as in previously described embodiments (such as that of FIG. 12), this embodiment could alternatively employ a rotating tube located within the sheath 212 and coupled to the mirrors to provide this function.

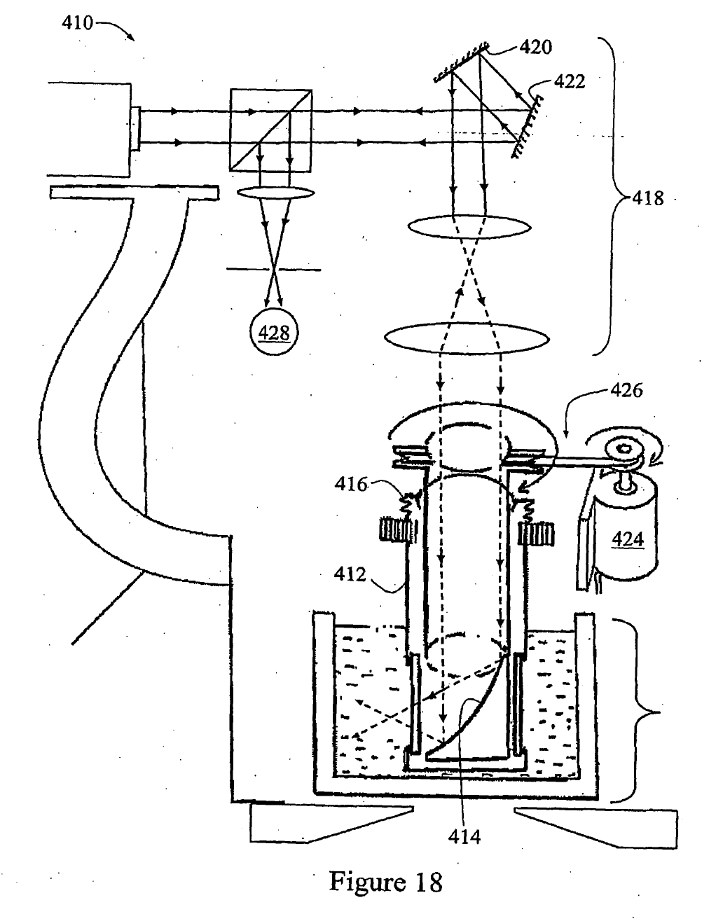

FIG. 18 is a schematic view of a scanning confocal microscope 410 according to a further embodiment of the invention. Microscope 410 includes a housing 412 and a mirror 414 in the form of an off-axis paraboloid of revolution mounted in the housing 412. The housing has an RMS thread 416 so that it is adapted to be screwed into the nose piece of a conventional microscope with single point laser scanning confocal microscope (LSCM) attachment 418.

The scanning mirrors 420, 422 of the LSCM attachment 418 are not operated; rather, images are built up by rotation of the mirror 414 using a motor 424, a belt drive 426 and constant speed gears.

A line scan is built up from the output(s) of the photodetector 428 of the LSCM attachment 418. As will be appreciated by those in the art, the Y motion of an imaging scan can be obtained by a number of possible mechanisms, such as a piezo-electrically actuated device located to press a resilient pad against a very fine thread in the rotating metal barrel of the mirror mount.

Synchronisation of the fast scan can be achieved by a number of means, such as a system in which a light beam projected by an LED towards a photodetector is interrupted by a projection from a collar which is rotated with the mirror mount.

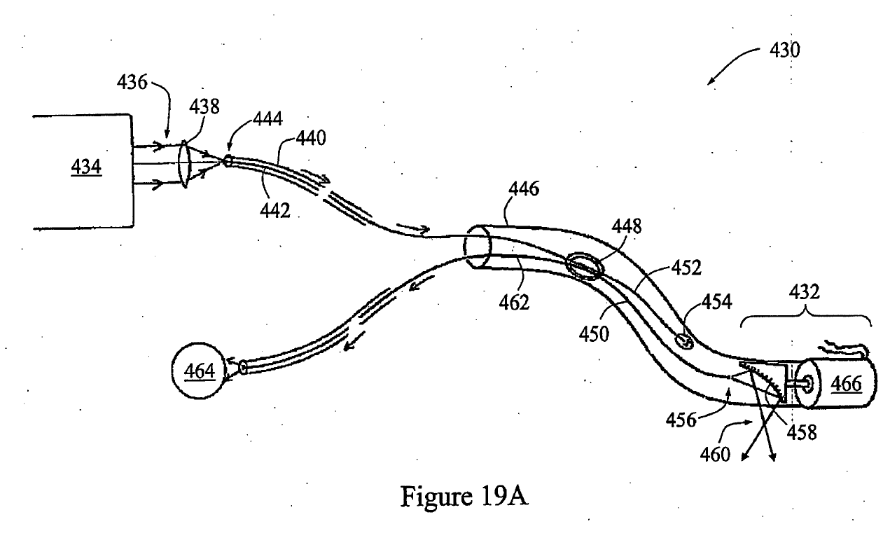

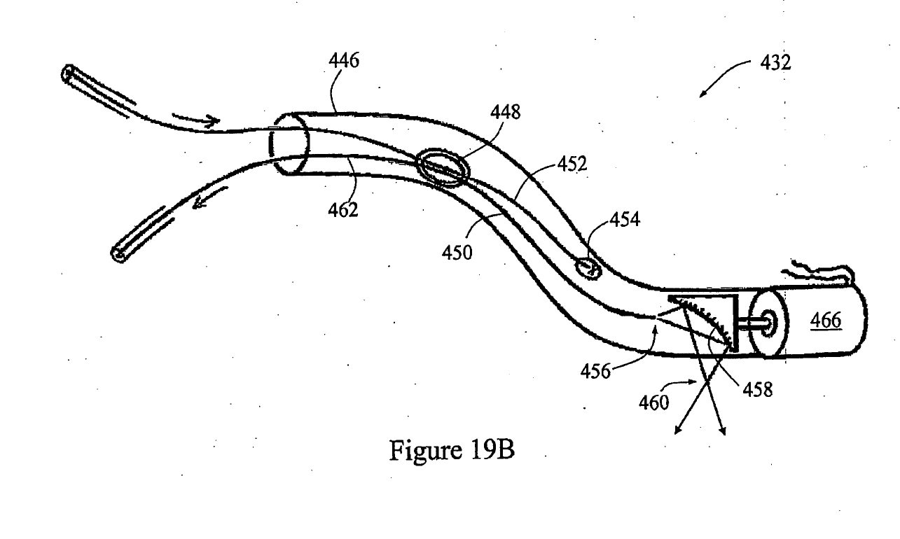

FIG. 19A is a schematic view of a scanning confocal endomicroscope 430 according to still another embodiment of the present invention. FIG. 19B is an enlarged view of the optical head 432 of endomicroscope 430. The endomicroscope 430 includes a laser source 434 for providing laser excitation light 436, a lens 438 and an optical fibre 440. The lens 438 is located to couple the light 436 into the core 442 at the proximal end 444 of fibre 440.

The endomicroscope 430 also includes a transparent sheath 446 that houses the fibre 440 for most of the fibre's length and the optical head 432, and an FBTC 448—to which the fibre 440 is coupled—located in the sheath 446. The FBTC 448 receives the light 436 from fibre 440 and splits it between first and second output fibre legs 450 and 452. The light in second fibre leg 452 is absorbed by index matching material 454 located at the distal end of the second fibre leg 452, while the light in first fibre leg 450 is emitted from the distal tip 456 of the first fibre leg and is reflected by an ellipsoidal mirror 458 located in the optical head 432, passes through the transparent wall of the sheath 446 and is focussed to a point observational field 460 within a specimen.

Light re-emitted from this focus returns along the same path along (including mirror 458 and first fibre leg 450) to the FBTC 448 and, as it is of longer wavelength, the return fluorescence component is preferably returned along third fibre leg 462 to a photodetector 464.

Scanning is effected by a motor 466 located in the optical head 432, behind the mirror 458.

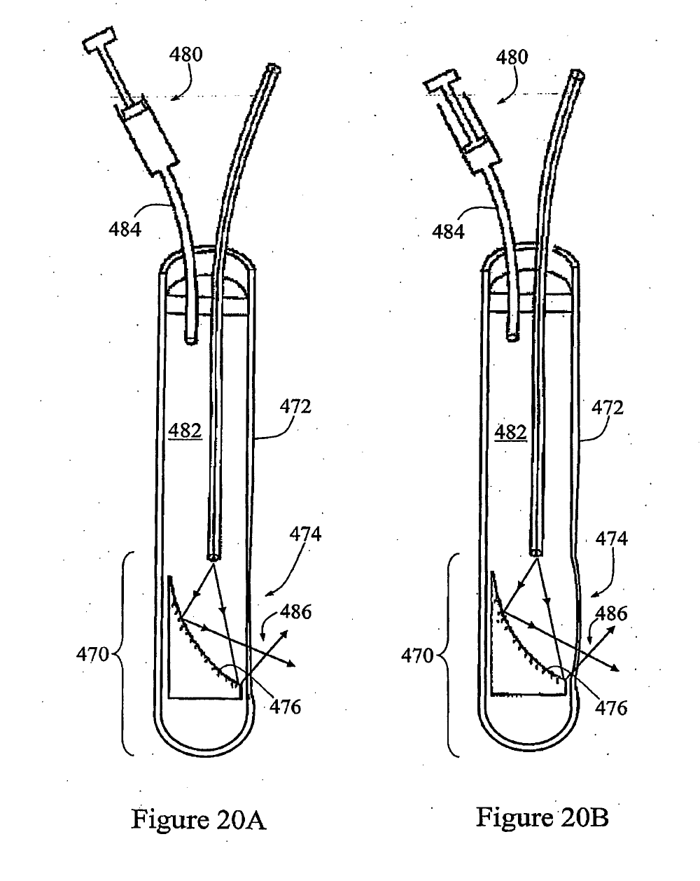

Some focussing methods have been discussed above, but a number of alternatives may also be employed. FIG. 20A is a schematic view of an optical head 470 for use in the above described embodiments, in which the external jacket 472 has a thin region in the form of a flexible membrane 474 coinciding with where excitation light is reflected through the jacket (as described above) by—in this example—ellipsoid mirror 476. Because it is thin, flexible membrane 474 requires a positive hydrostatic pressure inside to maintain its shape against the pressure exerted by a specimen into or against which the optical head 470 has been placed. If the pressure inside the jacket 472 is further increased, the thin region 474 of the jacket swells or deforms outwardly (as shown in FIG. 20B).

To control the internal pressure, a piston/cylinder mechanism 480 is provided, in fluid communication with the interior volume 482 of the optical head 470 by means of a conduit 484. Depressing the piston/cylinder mechanism 480 increases the pressure in the interior volume 482, which urges the flexible membrane 474 outwardly thereby pushing the specimen (typically of yielding tissue) out so that the point observational field 486 is closer to the surface of the specimen, as the distance from the surface of the jacket 472 to the point observational field 486 is reduced.

The thinness of the flexible membrane 474 is important, additionally, as it reduces the extent to which the optical path of the excitation light is disturbed. If this approach is used in a scanning system, the flexible membrane can be extended around the circumference of the optical head, though with ribs to prevent the overall length of the optical from increasing when the interior pressure is increased.

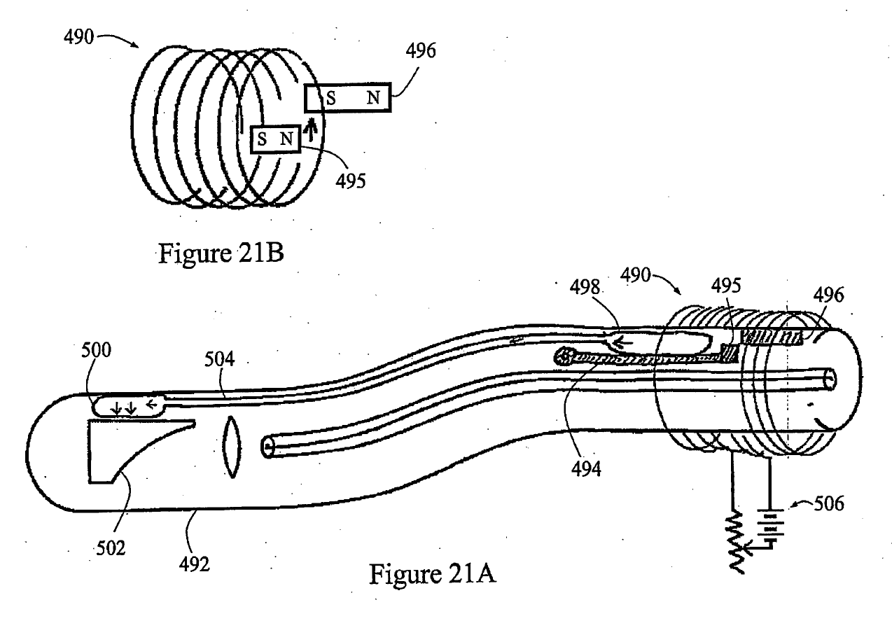

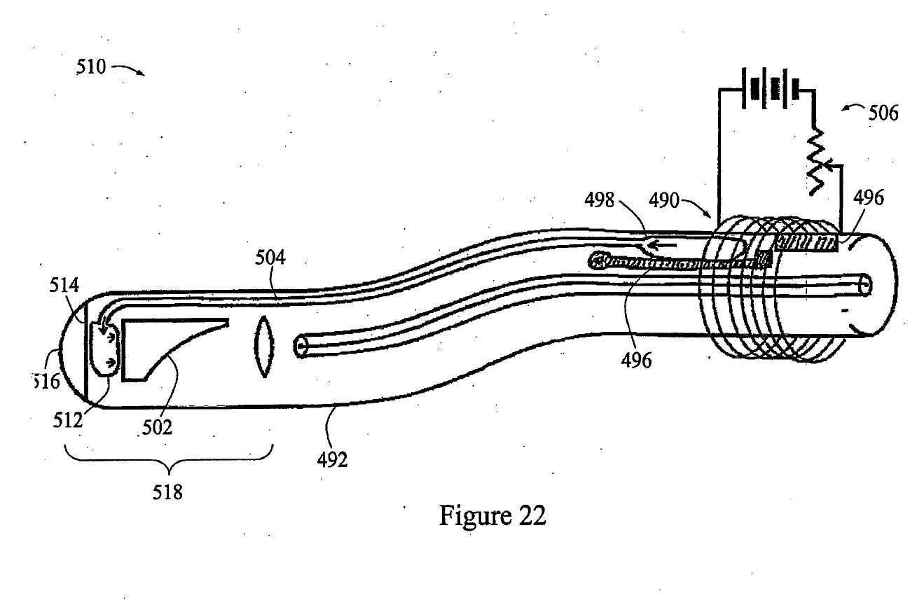

A hydraulic method by means of which Z motion (focussing) or Y motion (Y scan) could be made to occur in a rotating endomicroscope, as illustrated schematically in FIGS. 21A, 21B and 22. Referring to FIG. 21A, which shows a detail of such an endomicroscope, a coil of wire 490 is disposed around the rotating endoscope tube 492. The passage of electric current through this coil 490 causes an armature 494 (provided with a permanent magnet 495) within the tube 492 to be attracted to a polepiece 496, which compresses a first fluid-filled impermeable sack 498 located between the armature 494 and the tube 492. This compresses the fluid in the sack 498. The first sack 498 is in fluid communication with a second impermeable sack 500 located behind the mirror 502 via conduit 504, so the compression of the first sack 498 causes the second sack 500 to expand and urge the mirror 502 forwardly towards the wall of the tube. Consequently, the point observational field is moved to be at a greater depth within the specimen, and so Z scanning is effected.

The current in coil 490 is provided by variable power supply 506. The relative positions of the coil 490, permanent magnet 495 and polepiece 496 are shown in greater detail in FIG. 21B.

FIG. 22 is a schematic view of an apparatus for using a comparable hydraulic actuator as that shown in FIG. 21A, but arranged to effect a Y scan in a rotating scan mechanism. It is clear that the mechanisms of both FIGS. 21A and 22 could be made to operate by having coils located at different positions along the endoscope tube. The arrangement 510 of FIG. 22 is generally identical with that of FIG. 21A, except that a second impermeable sack 512 is located between the mirror 502 and a support plate 514 mounted on the distal side of the mirror 502 (and hence closer to the tip 516 of the optical head 518). Forcing fluid into second sack 512 urges the mirror in the Y direction, so that a scan can be obtained in that axis.

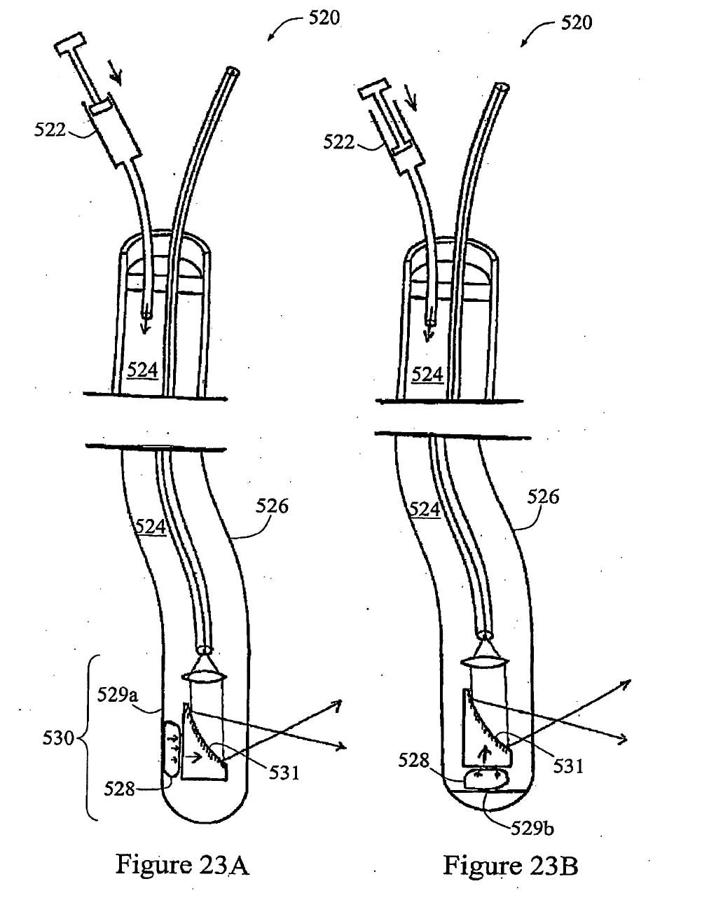

The piston/cylinder mechanism 480 of the embodiment of FIGS. 20A and 20B can also be used to actuate a sack comparable to the second sack 500 of FIG. 21A or second sack 514 of FIG. 22 respectively. FIG. 23A is a schematic view of an optical head 520 for use in the above described embodiments. A piston/cylinder mechanism 522 is used to inject or withdraw an incompressible fluid into the interior 524 of the endoscope tube 526, in order to compress or expand a small sealed bag or sack containing a compressible gas. FIG. 23A demonstrates how this can be used to effect a Z scan, by locating the bag or sack 528 between a aide wall 529a of the optical head 530 and the mirror 531. FIG. 23B demonstrates how this approach can be used to effect a Y scan, by locating the bag or sack 528 between the end wall 529b of the optical head 530 and the mirror 531.

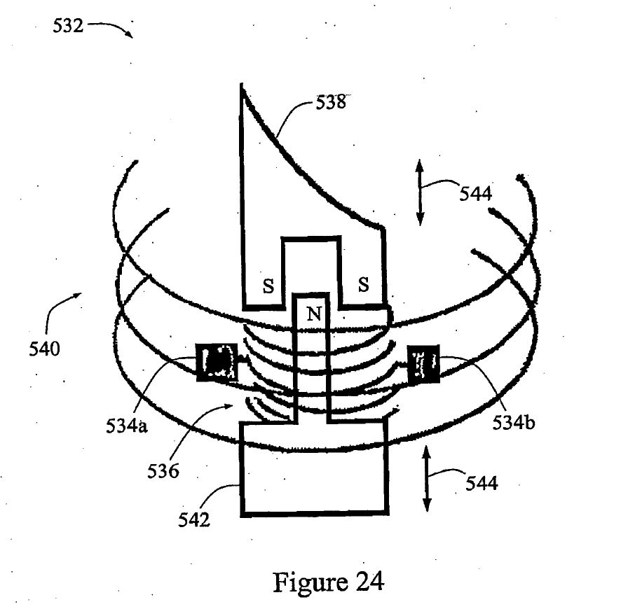

Alternatively, coils of wire wound around the tube of the endoscope can also be used to produce resonant motion of the mirror to effect scanning. FIG. 24 illustrates such a longitudinal resonant scan motion system 532 according to an embodiment of the invention, for use with the scanning apparatuses described above. Counterbalance masses 534a and 534b are provided, connected via a spring 536 to a mirror 538. Pulsed electric current in a coil 540 causes the mirror 538 and the counterweight 542 to mutually attract and oscillate in direction 544.

The spring 536 is held to the endoscope tube by its midpoint. Rotary oscillatory motion can be produced in a similar manner. A similar method to that illustrated by means of the embodiment of FIG. 24 could be used with a steady current to produce a scan in the Y direction.

Modifications within the scope of the invention may be readily effected by those skilled in the art. It is to be understood, therefore, that this invention is not limited to the particular embodiments described by way of example hereinabove.

In the following claims and in the preceding description of the invention, except where the context requires otherwise owing to express language or necessary implication, the word “comprise” or variations such as “comprises” or “comprising” is used in an inclusive sense, i.e. to specify the presence of the stated features but not to preclude the presence or addition of further features in various embodiments of the invention.

Further, any reference herein to prior art is not intended to imply that such prior art forms or formed a part of the common general knowledge.

1. A microscope or endoscope, comprising:
a light source;
a flexible light transmitter for receiving and transmitting excitation light from said light source;
an optical element with a forward end for receiving said excitation light from said light transmitter and a rear wall having an internal surface for reflecting said excitation light laterally; and
an external sleeve enclosing said optical element and transparent to said excitation light in at least a region of said sleeve where said light is directed by said internal surface;
wherein said internal surface has an optical figure suitable for focussing said excitation light to a point observational field external to said sleeve.

2. A microscope or endoscope as claimed in claim 1, wherein the optical element is rotatable, and said microscope or endoscope includes a drive coupled to said optical element, for rotating said optical element so that said point observational field can be scanned.

3. A microscope or endoscope as claimed in claim 2, wherein the drive comprises an electrical motor located external to said optical element and adjacent to said rear surface of said optical element.

4. A microscope or endoscope as claimed in claim 2, wherein the drive comprises an inner sleeve for supporting and rotating said optical element, whereby said light can be scanned relative to a sample by rotating said inner sleeve and thereby the direction in which said excitation light is focussed by said internal surface.

5. A microscope or endoscope as claimed in claim 4, wherein the drive includes an electrical or other motor for rotating the inner sleeve.

6. A microscope or endoscope as claimed in claim 1, wherein the internal surface is a section of an ellipsoid, whereby the optical element is adapted to receive said light from a substantially point source.

7. A microscope or endoscope as claimed in claim 1, wherein the internal surface is a section of a paraboloid, whereby the optical element is adapted to receive said excitation light in substantial parallel form.

8. A microscope or endoscope as claimed in claim 1, wherein said optical element includes at least one further optical element for changing said excitation light from a divergent beam into a substantial parallel beam, and the internal surface is a section of a paraboloid.

9. A microscope or endoscope as claimed in claim 1, wherein the internal surface has a reflective coating to increase the efficiency of reflection.

10. A microscope or endoscope as claimed in claim 1, wherein the optical element is oriented with the rear wall proximal to said light source.

11. A microscope or endoscope as claimed in claim 10, wherein the flexible light transmitter includes a bend in order to direct said excitation light towards said internal surface.

12. A microscope or endoscope as claimed in claim 10, further including a mirror for receiving said light from said flexible light transmitter and directing said excitation light towards said internal surface of said optical element.

13. A microscope or endoscope as claimed in claim 1, further including a short coherence length light source and a beamsplitter, for performing simultaneous optical coherence tomography.

14. A microscope or endoscope as claimed in claim 1, provided with or as a hand-held probe housing said optical element.

15. An optical head for an endoscope or microscope, comprising:
an optical element with a forward end for admitting excitation light from a light source, and a rear wall having an internal surface for reflecting said excitation light laterally, wherein said internal surface has an optical figure suitable for focussing said excitation light; and
an external sleeve enclosing said optical element and transparent to said excitation light in at least a region of said sleeve where said excitation light is directed by said internal surface.

16. An optical head as claimed in claim 15, wherein the optical element is rotatable, and said head includes a drive coupled to said optical element, for rotating said optical element so that said point observational field can be scanned.

17. An optical head as claimed in claim 15, further including a beamsplitter for separating out light from a short coherence length light source, for performing simultaneous optical coherence tomography.

18. An optical head as claimed in claim 15, provided in a hand-held probe.

19. An optical element comprising:
a forward and for admitting light from a light sources
a rear wall having an internal surface for reflecting said light laterally; and
an external sleeve of non-uniform thickness;
wherein said internal surface has an optical figure suitable for focussing said light to a point observational field outside said sleeve, said external sleeve is rotatable, translatable or both rotatable and translatable relative to said optical element whereby the distance of said point from said Sleeve can be varied, and said external sleeve is transparent to said light in at least the region where said light is directed by said internal surface.

20. An optical element as claimed in claim 19, further including a beamsplitter for separating out light from a short coherence length light source, for performing simultaneous optical coherence tomography.

21. An optical element as claimed in claim 19, provided in a hand-held probe.

22. A microscope or endoscope as claimed in claim 1, wherein the external sleeve is composed of a polymer with a refractive index identical with or comparable to an expected specimen in at least a region of said external sleeve where said excitation light is directed by said internal surface, for transmitting said light and return light.

23. An optical head as claimed in claim 15, wherein the external sleeve is composed of a polymer with a refractive index identical with or comparable to an expected specimen in at least a region of said external sleeve where said excitation light is directed by said internal surface, for transmitting said light and return light.

24. An optical element as claimed in claim 19, wherein the external sleeve is composed of a polymer with a refractive index identical with or comparable to an expected specimen in at least a region of said external sleeve where said excitation light is directed by said internal surface, for transmitting said light and return light.

25. A microscope or endoscope as claimed in claim 1, wherein the optical element is filled with a liquid that has a refractive index that matches, or is close to, the refractive index of the tissue under examination, in order to minimize refraction of light rays in passing from the interior of the block to a point observational field within a specimen.

26. A optical coherence tomograph, comprising:
a short coherence length light source;
a flexible light transmitter for receiving and transmitting light from said light source;
an optical element with a forward end for receiving said light from said light transmitter and a rear wall having an internal surface for reflecting said light laterally; and
an external sleeve enclosing said optical element and transparent to said light in at least a region of said external sleeve where said light is directed by said internal surface;
wherein said internal surface has an optical figure suitable for focussing said excitation light to an observational field external to said external sleeve.

27. A method of performing microscopy, endoscopy or optical coherence tomography, comprising:
locating an optical element in or adjacent a specimen, the optical element having a forward end for receiving excitation light from a light transmitter and a rear wall having an internal surface for reflecting said excitation light laterally with an optical figure suitable for focussing said excitation light to an observational field in or on said specimen;
directing excitation light against said internal surface and thereby to said observation field; and
collecting by means of said internal surface return light emitted from said specimen in response to said excitation light.

28. A method as claimed in claim 27, wherein the optical element is located in an external sleeve that is transparent to said excitation light in at least a region of said sleeve where Said excitation light is directed by said internal surface.

29. A method as claimed in claim 27, further including rotating said optical element, thereby effecting scanning.
